(19) United States
(12) Patent Application Publication
(10) Pub. No.: US 2018/0215783 A1
(43) Pub. Date: Aug. 2, 2018

(54) BIOORTHOGONAL REACTION OF AN AMINE N-OXIDE AND A BORON AGENT

(71) Applicant: The Regents of the University of California, Oakland, CA (US)

(72) Inventors: Carolyn R. Bertozzi, Stanford, CA (US); Justin Kim, Berkeley, CA (US)

(21) Appl. No.: 15/747,129

(22) PCT Filed: Aug. 4, 2016
(86) PCT No.: PCT/US16/45651

§ 371 Date: Jan. 23, 2018

Related U.S. Application Data
(60) Provisional application No. 62/204,883, filed on Aug. 13, 2015.

(51) Int. Cl. C07K 1/107; C07K 1/13; A61K 47/68; A61K 47/66; A61K 47/54; A61K 47/18; C07F 5/02

(52) U.S. Cl. CPC C07K 1/107; C07K 1/13; A61K 47/6891; G01N 33/532; A61K 47/558; A61K 47/18; C07F 5/025; A61K 47/66

(57) ABSTRACT

Methods for chemoselective modification of a target molecule comprising an amine N-oxide in a biological sample are provided. Aspects of the methods include selectively reacting the amine N-oxide group of the target molecule with a boron agent, where the reacting reduces the amine N-oxide to an amine to produce a modified target molecule. Modification of the target molecule using the subject methods may produce an activated target molecule, e.g., a detectable or bioactive. In some cases, chemoselective modification leads to cleavage of the modified target molecule to produce a first target fragment and a second target fragment. Also provided are compositions useful in practicing various embodiments of the subject methods.

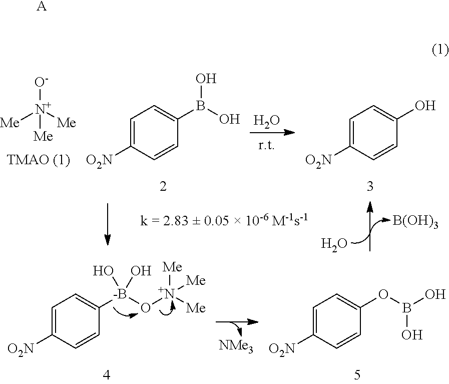

### CROSS-REFERENCE

This application claims the benefit of U.S. Provisional Patent Application No. 62/204,883, filed Aug. 13, 2015, which application is incorporated herein by reference in its entirety.

### STATEMENT REGARDING FEDERALLY SPONSORED RESEARCH

This invention was made with government support under Grant No. GM058867 awarded by The National Institutes of Health. The government has certain rights in the invention.

### INTRODUCTION

The field of bioorthogonal chemistry strives to meet the demands for tools that can facilitate the molecular analysis of biological processes. Its foundational applications were in the chemical targeting of biomolecules with probes and affinity reagents, both in cultured cells and live organisms, and in building hybrid biomolecule constructs for therapeutic applications. A reaction compendium of bioorthogonal chemistries that are available includes the Staudinger ligation, Pictet-Spengler ligation, copper-mediated and metal-free azide-alkyne cycloadditions, tetrazine ligations, and nitrile oxide olefin cycloaddition.

### SUMMARY

Methods for chemoselective modification of a target molecule comprising an amine N-oxide in a biological sample are provided. Aspects of the methods include selectively reacting the amine N-oxide group of the target molecule with a boron agent, where the reacting reduces the amine N-oxide to an amine to produce a modified target molecule. Modification of the target molecule using the subject methods may produce an activated target molecule, e.g., a detectable or bioactive. In some cases, chemoselective modification leads to cleavage of the modified target molecule to produce a first target fragment and a second target fragment. Also provided are compositions useful in practicing various embodiments of the subject methods.

The present disclosure provides a method for chemoselective modification of a target molecule comprising an amine N-oxide in a biological sample, the method comprising: selectively reacting the amine N-oxide group of the target molecule with a boron agent, wherein the reacting reduces the amine N-oxide to an amine to produce a modified target molecule.

In some embodiments, the modified target molecule is an activated target molecule. In some cases, the modified target molecule is a detectable target molecule or a bioactive target molecule. In some embodiments, the modified target molecule is cleaved to produce a first target fragment and a second target fragment.

In some embodiments, the target molecule comprises a profluorophore and the reacting activates the profluorophore to produce a fluorescent target molecule. In some embodiments, the target molecule comprises a prodrug and the reacting activates the prodrug to produce a drug. In some embodiments, the reacting modifies an amine N-oxide-linker of the target molecule to produce a cleavable amine-linker.

In some embodiments, the target molecule comprises a biomolecule covalently linked via the amine N-oxide-linker to a chemical entity and the method further comprises cleaving the cleavable amine-linker to release the chemical entity. In some embodiments, the chemical entity is a drug or a detectable label. In some embodiments, the amine N-oxide-linker comprises a self immolative linker group. In some embodiments, the amine N-oxide-linker comprises a group selected from the group consisting of para-amino-benzyloxycarbonyl (PABC), meta-amino-benzyloxycarbonyl (MABC), para-amino-benzyloxy (PABO), meta-amino-benzyloxy (MABO) and para-aminobenzyl.

In some embodiments, the target molecule comprises a biomolecule. In some embodiments, the biomolecule is a protein. In some embodiments, the biomolecule is an antibody or an antibody fragment.

In some embodiments, the boron agent has the formula

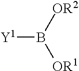

wherein: Y¹ is an aryl, a substituted aryl, an alkyl, a substituted alkyl or —B(OR⁶)(OR⁷); R¹, R², R⁶ and R⁷ are each independently H, an alkyl, a substituted alkyl, and R¹ and R² or R⁶ and R⁷ may be optionally cyclically linked. In some embodiments, Y¹ is —B(OR⁶)(OR⁷). In some embodiments, the boron agent is a diboron agent. In some embodiments, the boron agent is bis(pinacolato)diboron ((Bpin)₂) or bis(catecholato)diboron).

In some embodiments, the biological sample comprises a cell, a cell lysate, a tissue or a fluid. In some embodiments, the biological sample is in vivo.

The present disclosure provides a composition, comprising: a target molecule comprising an amine N-oxide; and a boron agent; contained in a biological sample.

The present disclosure provides a conjugate, comprising a first target molecule and a second target molecule, covalently linked via an amine N-oxide-linker. In some embodiments, the first target molecule is a biomolecule. In some embodiments, the second target molecule is a biomolecule. In some embodiments, the second target molecule is a chemical entity. In some embodiments, the chemical entity is a drug or a detectable label. In some embodiments, the biomolecule is an antibody or an antibody fragment and the chemical entity is a chemotherapeutic drug. In some embodiments, the amine N-oxide-linker comprises a self immolative linker. In some embodiments, the amine N-oxide-linker comprises a group selected from the group consisting of para-amino-benzyloxycarbonyl (PABC), meta-amino-benzyloxycarbonyl (MABC), para-amino-benzyloxy (PABO), meta-amino-benzyloxy (MABO) and para-aminobenzyl.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 illustrates the characterization of kinetic parameters of embodiments of the subject methods. (A) The reaction between trimethylamine N-oxide (TMAO) (1) and p-nitrophenylboronic acid (2) features biologically compatible reagents and byproducts. (B) Reaction of N,N-dialkylaniline-derived N-oxide 6 and phenylboronic acid is three orders of magnitude faster.

FIG. 2 illustrates the results of experiments demonstrating the bioorthogonality of the subject methods. (A) B₂pin₂ rapidly reduces N-oxide 6 in phosphate buffered saline (PBS) (pH 7.4). The second-order rate constant was calculated from fluorescence measurements of fluorophore 7 under pseudo-first-order conditions with saturating concentrations of B₂pin₂. (B) Steric relaxation around the N-oxide enhances the reaction rate, and the second-order rate constant exceeds 10³ M⁻¹s⁻¹. (C) N-oxide 9 conjugated to a 34 kDa HaloTag protein rapidly reacts with B₂pin₂ in PBS. Reaction conversion for the lowest and highest B₂pin₂ concentrations used in kinetics experiments are displayed. Data represent the mean of experiments performed in triplicate. The vertical dotted lines trace out the half-lives of the reactions at the respective saturating concentrations of B₂pin₂. (D) Bis-N-oxide profluorophore 10 was conjugated to a 64 kDa GFP-HaloTag fusion protein and treated with stoichiometric to slightly superstoichiometric quantities of B₂pin₂. The red and green channels represent TAMRA and GFP signals, respectively. (E) N-oxide profluorophore 6 was reacted with 0, 5, 50, or 500 μM (columns 1-4 from left to right) B₂pin₂ in Jurkat and E. coli cell lysates consisting of 1 mg/mL protein and then normalized to the level of a positive control consisting of fluorophore 7 (right most column 5). Data represent the mean of experiments performed in triplicate. Error bars represent a standard deviation. (F) Time-dependent fluorescence measurements reveal the stability of N-oxide profluorophore 6 to mammalian cell lysate. Data represent the mean of experiments performed in triplicate. (G) Viability of three mammalian cell lines in a range of B₂pin₂ concentrations was evaluated after 24 h using an MTT assay. Data represent the mean of experiments done in triplicate. Error bars represent a standard deviation.

FIG. 3 illustrates the results of experiments demonstrating the subject N-oxide-boron agent methods are compatible with common bioorthogonal reactions. (A) HEK293T cell surfaces were modified with aldehydes, N-oxides, azides, or cyclopropenes, mixed, then treated with a cocktail of bioorthogonal reagents. Each population was analyzed by flow cytometry for reaction with aminooxy-Alexa Fluor 488 (left column 1), B₂pin₂ (from left column 2), DIBAC-Cy5 (from left column 3), and tetrazine-Cy7 (right most column 4). The mean normalized fluorescence intensity of each fluorophore from three biological replicates is plotted. Error bars represent a standard deviation. (B) N-oxide-modified HEK293T cells were treated with aminooxy, DIBAC, and tetrazine reagents with or without diboron. N-oxides are not reactive toward hydroxylamines, cyclooctynes, or tetrazines.

FIG. 4 shows fluorescence microscopy images demonstrating that B₂pin₂ activates N-oxides on cytoplasmic proteins inside mammalian cells. HEK293T cells transiently transfected with GFP-HaloTag protein were incubated with 100 μM fluorophore 10, washed, treated with 0 or 100 μM B₂pin₂, then imaged by confocal microscopy after 45 min. Green (left column), red (middle column), and blue (not shown) channels represent GFP, TAMRA, and Hoechst 33342 fluorescence, respectively. The merged image (right column) is a composite of all three channels with a phase contrast image.

### DEFINITIONS

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

As used herein, the terms “amine N-oxide”, “N-oxide” and “amine oxide” are used interchangeably and refer to a chemical group that contains an N—O bond with three additional hydrogen and/or substituent groups attached to N. The functional group may be represented in a variety of ways, such as R₃N⁺—O or R₃N→O where each R is independently hydrogen or an amine substituent group (e.g., as described herein).

As used herein, the term “bioorthogonal” refers to a chemical reaction that can occur in a biological system or sample with interfering with native biochemical processes, i.e., without cross-reactivity to functional groups and chemistries present in the system or sample.

As used herein, the term “linker” or “linkage” refers to a linking moiety that connects two groups and, in some cases, has a backbone of 100 atoms or less in length. A linker or linkage may be a covalent bond that connects two groups or a chain of between 1 and 100 atoms in length, for example a chain of 1, 2, 3, 4, 5, 6, 8, 10, 12, 14, 16, 18, 20, 30, 40 or 50 carbon atoms or less in length, where the linker may be linear, branched, cyclic or a single atom. In certain cases, one, two, three, four or five or more carbon atoms of a linker backbone may be optionally substituted with a sulfur, nitrogen or oxygen heteroatom. The bonds between backbone atoms may be saturated or unsaturated, and in some cases not more than one, two, or three unsaturated bonds are present in a linker backbone. The linker may include one or more substituent groups, for example with an alkyl, aryl or alkenyl group. A linker may include, without limitations, polyethylene glycol (PEG), including modified PEG groups; ethers, thioethers, tertiary amines, alkyls, which may be straight or branched, e.g., methyl, ethyl, n-propyl, 1-methylethyl (isopropyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term “fusion protein” or grammatical equivalents thereof is meant to include a protein composed of a plurality of polypeptide components, that while typically unjoined in their native state, typically are joined by their respective amino and carboxyl termini through a peptide linkage to form a single continuous polypeptide. Fusion proteins may be a combination of two, three or even four or more different proteins. The term polypeptide includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

In general, polypeptides may be of any length, e.g., 2 or greater amino acids, greater than 4 amino acids, greater than about 10 amino acids, greater than about 20 amino acids, greater than about 50 amino acids, greater than about 100 amino acids, greater than about 300 amino acids, usually up to about 500 or 1000 or more amino acids. “Peptides” are generally 2 or greater amino acids in length, such as greater than 4 amino acids, greater than about 10 amino acids, greater than about 20 amino acids, usually up to about 50 amino acids. In some embodiments, peptides are between 2 and 30 amino acids in length.

The term “capture agent” or “affinity capture agent” refers to an agent that binds an analyte through an interaction that is sufficient to permit the agent to extract and concentrate the analyte from a homogeneous mixture of different analytes. The binding interaction is typically mediated by an affinity region of the capture agent. Typical capture agents include antibodies, which are well known in the art. Capture agents usually “specifically bind” one or more analytes.

Thus, the term “capture agent” refers to a molecule or a multi-molecular complex which can specifically bind an analyte, e.g., specifically bind an analyte for the capture agent with a dissociation constant (K_{D}) of less than about 10⁻⁶ without binding to other targets.

The term “specific binding” refers to the ability of a capture agent to preferentially bind to a particular analyte that is present in a homogeneous mixture of different analytes. Typically, a specific binding interaction will discriminate between desirable and undesirable analytes in a sample, typically more than about 10 to 100-fold or more (e.g., more than about 1000-fold). Typically, the affinity between a capture agent and analyte when they are specifically bound in a capture agent/analyte complex is at least 10⁻⁸ M, at least 10⁻⁹ M, usually up to about 10⁻¹⁰ M.

The terms “antibody” and “immunoglobulin” are used interchangeably herein to refer to a capture agent that has at least an epitope binding domain of an antibody. These terms are well understood by those in the field, and refer to a protein containing one or more polypeptides that specifically binds an antigen. One form of antibody constitutes the basic structural unit of an antibody. This form is a tetramer and consists of two identical pairs of antibody chains, each pair having one light and one heavy chain. In each pair, the light and heavy chain variable regions are together responsible for binding to an antigen, and the constant regions are responsible for the antibody effector functions.

The recognized immunoglobulin polypeptides include the kappa and lambda light chains and the alpha, gamma (IgG₁, IgG₂, IgG₃, IgG₄), delta, epsilon and mu heavy chains or equivalents in other species. Full-length immunoglobulin “light chains” (of about 25 kDa or about 214 amino acids) comprise a variable region of about 110 amino acids at the NH₂-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions, e.g., gamma (of about 330 amino acids).

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like. Also encompassed by the terms are Fab′, Fv, F(ab′)₂, and or other antibody fragments that retain specific binding to antigen.

Antibodies may exist in a variety of other forms including, for example, Fv, Fab, and (Fab′)₂, as well as bi-functional (i.e. bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., “Immunology”, Benjamin, N.Y., 2nd ed. (1984), and Hunkapiller and Hood, Nature, 323, 15-16 (1986). Monoclonal antibodies and “phage display” antibodies are well known in the art and encompassed by the term “antibodies”.

As used herein, “binding partners” and equivalents refer to pairs of molecules that can be found in a capture agent/analyte complex, i.e., exhibit specific binding with each other.

As used herein, the term “a target protein” refers to all members of the target family, and fragments and enantiomers thereof, and protein mimics thereof. The target proteins of interest that are described herein are intended to include all members of the target family, and fragments and enantiomers thereof, and protein mimics thereof, unless explicitly described otherwise. The target protein may be any protein of interest, such as a therapeutic or diagnostic target, including but not limited to: hormones, growth factors, receptors, enzymes, cytokines, osteoinductive factors, colony stimulating factors and immunoglobulins. The term “target protein” is intended to include recombinant and synthetic molecules, which can be prepared using any convenient recombinant expression methods or using any convenient synthetic methods, or purchased commercially, as well as fusion proteins containing a target molecule.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “substituted alkyl” refers to an alkyl group as defined herein wherein one or more carbon atoms in the alkyl chain (except the C₁ carbon atom) have been optionally replaced with a heteroatom such as —O—, —N—, —S—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R′ and R″ may be the same or different and are chosen from hydrogen, optionally substituted alkyl, cycloalkyl, alkenyl, cycloalkenyl, alkynyl, aryl, heteroaryl and heterocyclic.

“Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.

“Amino” refers to the group —NH₂.

The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂ M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a boron reagent” includes a plurality of such reagents and reference to “the N-oxide moiety” includes reference to one or more N-oxide moieties and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

As summarized above, aspects of the present disclosure include a method for chemoselective modification of a target molecule in a biological sample by selective reaction of an amine N-oxide and a boron agent.

The subject methods provide a bioorthogonal reaction between an amine N-oxide functional group and a boron agent to reduce the amine N-oxide to an amine, as depicted in Scheme 1 below:

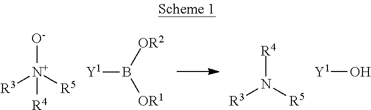

where Y¹ and R¹-R⁵ may be any convenient groups. In some cases, Y¹ is an aryl, a substituted aryl, a heteroaryl, a substituted heteroaryl, an alkyl, a substituted alkyl or —B(OR⁶)(OR⁷); and R¹-R⁷ are each independently H, an alkyl, a substituted alkyl, an aryl, a substituted aryl, a heteroaryl or a substituted heteroaryl, where R¹ and R² or R⁶ and R⁷ may be optionally cyclically linked.

The subject methods are bioorthogonal such that the subject amine N-oxide/boron reaction can be performed in the presence of the endogenous components of any convenient biological sample. In addition, the subject methods are also orthogonal to a variety of other non-naturally occurring chemistries which find use in biological systems. For example, the amine N-oxide-boron chemistry described herein is orthogonal to (i.e., is not cross-reactive with) chemistries involving, e.g., hydroxylamines, cyclooctynes or tetrazines. The subject methods provide for selective reduction of an amine N-oxide group using a boron agent in a complex biological system or sample.

The amine N-oxide reduction reaction described herein may be performed chemoselectively in the biological sample to produce the products shown in scheme 1. The amine N-oxide and boron agent components of the subject method may be configured in any convenient way to adapt the reaction to a desired application. By incorporation of these components into target molecules, the selective manipulation of molecules, cells, particles and surfaces can be achieved, including the manipulation, release, tagging and tracking of biomolecules in vitro and in vivo. For example, by incorporation of the amine N-oxide into a target molecule of interest, the target molecule may be selectively modified to an amine in situ in a biological sample of interest. The chemoselective modification of a target molecule to produce an amine functional group can be configured to provide for a desirable change in the biological sample, e.g., a fluorogenic response, activation of a biological activity, selective cleavage and release of a cargo moiety from a conjugate.

In some embodiments, the subject methods include introducing an amine-N-oxide containing target molecule (e.g., as described herein) to a biological sample. In some embodiments, the subject method is an in vitro method that includes contacting a biological sample with an amine-N-oxide containing target molecule in conjunction with a boron agent. The boron agent can be added to the biological sample at a particular time and in a particular effective amount to result in the chemoselective modification of the target molecule. The subsequent effects on the biological sample of the modified target molecule can then be observed using any convenient methods. The protocols that may be employed in these methods are numerous. Assays which may be adapted for use in the subject methods include, but are not limited to, cell-free assays, binding assays, cellular assays in which a cellular phenotype is measured, e.g., gene expression assays and and assays that involve a particular animal model for a condition of interest.

In some embodiments, the method includes selectively reacting the amine N-oxide group of the target molecule with a boron agent, wherein the reacting reduces the amine N-oxide to an amine to produce a modified target molecule. The modified target molecule may be an activated target molecule, e.g., activated to have a desirable spectroscopic property or biological activity relative to the target molecule. In some cases, the target molecule is fluorogenic and the desirable spectroscopic property of the modified target molecule is fluorescence. In certain embodiments, the method further includes detecting, directly or indirectly, the modified target molecule. In certain instances, modified target molecule is a conjugate and the method further includes cleaving the modified target molecule to produce a first target fragment (e.g., a polypeptide) and a second target fragment (e.g., a cargo moiety) (e.g., as described herein).

### Boron Agents

Any convenient boron-containing compound may be adapted for use as a boron agent in the subject methods and compositions. In some cases, the boron agent is a boronic acid. In some cases, the boron agent is a boronic ester. In certain cases, the boron agent is a diboron agent, e.g., a diborane compound. Any convenient diborane compounds may be adapted for use as a boron agent in the subject methods and compositions.

In some embodiments, the boron agent has the formula

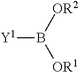

wherein:
Y¹ is an aryl, a substituted aryl, a heteroaryl, a substituted heteroaryl, an alkyl, a substituted alkyl, an alkoxy, a substituted alkoxy or —B(OR⁶)(OR⁷);
R¹, R², R⁶ and R⁷ are each independently H, an alkyl, a substituted alkyl, an aryl, a substituted aryl, a heteroaryl or a substituted heteroaryl, and
R¹ and R² or R⁶ and R⁷ may be optionally cyclically linked.

In certain embodiments, Y¹ is an aryl, a substituted aryl, a heteroaryl or a substituted heteroaryl. In certain embodiments, Y¹ is an alkyl or a substituted alkyl. In certain embodiments, Y¹ is an alkoxy or a substituted alkoxy.

In certain embodiments, R¹ and R² are independently an alkyl or a substituted alkyl. In certain embodiments, R¹ and R² are independently an aryl, a substituted aryl, a heteroaryl or a substituted heteroaryl. In certain embodiments, R¹ and R² are cyclically linked. In certain embodiments, R¹ and R² are linked to form a divalent boron substituent. In certain embodiments, R¹ and R² together with the oxygen atoms to which they are attached comprise a pinacol.

In certain embodiments, Y¹ is —B(OR⁶)(OR⁷). In certain embodiments, R⁶ and R⁷ are independently an alkyl or a substituted alkyl. In certain embodiments, R⁶ and R⁷ are independently an aryl, a substituted aryl, a heteroaryl or a substituted heteroaryl. In certain embodiments, the boron agent is a tetraalkoxydiboron. In certain embodiments, the boron agent is ((Bpin)₂). In certain embodiments, the boron agent is bis(catecholato)diboron).

Boron agents of interest include, but are not limited to, bis(pinacolato)diboron, bis(catecholato)diboron and tetrahydroxydiboron.

### Biological Samples

The subject bioorthogonal methods may be performed in a variety of biological samples. As used herein, the term “a biological sample” refers to a whole organism or a subset of its tissues, cells or component parts (e.g. body fluids, including, but not limited to, blood, serum, plasma, bronchoalveolar lavage, mucus, lymphatic fluid, synovial fluid, cerebrospinal fluid, saliva, cerebrospinal fluid, amniotic fluid, amniotic cord blood, urine, vaginal fluid, and semen). A “biological sample” can also refer to a homogenate, lysate or extract prepared from a whole organism or a subset of its tissues, cells or component parts, or a fraction or portion thereof, including but not limited to, plasma, serum, spinal fluid, lymph fluid, the external sections of the skin, respiratory, intestinal, and genitourinary tracts, tears, saliva, milk, blood cells, tumors and organs. In certain embodiments, the sample has been removed from an animal or plant. Biological samples may include cells. The term “cells” is used in its conventional sense to refer to the basic structural unit of living organisms, both eukaryotic and prokaryotic, having at least a nucleus and a cell membrane. In certain embodiments, cells include prokaryotic cells, such as from bacteria. In other embodiments, cells include eukaryotic cells, such as cells obtained from biological samples from animals, plants or fungi. The sample may include a heterogeneous cell population from which target cells are isolated. In some instances, the sample includes peripheral whole blood, peripheral whole blood in which erythrocytes have been lysed prior to cell isolation, cord blood, bone marrow, density gradient-purified peripheral blood mononuclear cells or homogenized tissue.

In some embodiments, the biological sample includes a cell. A variety of cells may be used in conjunction with the subject methods. Target cells of interest include, but are not limited to, stem cells, e.g., pluripotent stem cells, hematopoietic stem cells, T cells, T regulator cells, dendritic cells, B Cells, e.g., memory B cells, antigen specific B cells, granulocytes, leukemia cells, lymphoma cells, virus-infected cells (e.g., HIV-infected cells), natural killer (NK) cells, macrophages, monocytes, fibroblasts, epithelial cells, endothelial cells, and erythroid cells. Target cells of interest include cells that have a convenient cell surface marker or antigen that may be captured by a convenient specific binding member or conjugates thereof.

### Amine N-Oxide Containing Target Molecules

Any convenient molecules of interest may be adapted for use in the subject methods and compositions. An amine-N-oxide functional group may be installed in a target molecule of interest in a variety of ways.

In some cases, a target molecule precursor includes an amine group which may itself be directly converted into an amine-N-oxide to produce the amine-N-oxide-containing target molecule. Procedures of interest which may be adapted to use in preparing an amine-N-oxide target molecule include those described by Niwa et al. (Org. Biomol. Chem. 2014, 12, 6590) and Hirayama et al. (Chem. Sci. 2013, 4, 1250). In some cases, the target molecule is an amine containing fluorophore which can be directly converted into an amine-N-oxide containing fluorogenic molecule. Chemoselective modification of the amine-N-oxide containing fluorogenic molecule by reaction with a boron agent converts the fluorogenic molecule back to a fluorophore and produces a fluorescent signal in the biological sample. Fluorophores of interest which may be configured to include an amine-N-oxide include, but are not limited to, rhodamine, Texas Red, tetramethylrhodamine, carboxyrhodamine, carboxyrhodamine 6G, carboxyrhodol, carboxyrhodamine 110, ROX (5-(and -6)-carboxy-X-rhodamine, and the like.

The amine-N-oxide containing fluorogenic molecule may then be conjugated to a second target molecule of interest (e.g., a biomolecule) to provide for the tagging and tracking of molecules in vitro and in vivo.

In some cases, the N-oxide functional group may be installed in a target molecule indirectly. For example, the N-oxide functional group can be attached to a target molecule of interest via a linker which includes an additional functional group capable of conjugation to the molecule of interest.

In some cases, the target molecule is endogenous to the biological sample and is adapted for use in the subject methods and compositions. In some cases, the target molecule is non-naturally occurring to the biological sample but is selected to provide for a desirable property which finds use in the subject methods.

Target molecules of interest include, but are not limited to, polypeptides (e.g., antibodies or peptides), chemical entities (e.g., detectable labels or drugs), nucleic acids, lipids (e.g., fatty acids), sugars (e.g., saccharides), steroids, purines, pyrimidines and water-soluble polymers, and derivatives, structural analogs, conjugates or combinations thereof. Any convenient combinations or conjugates of target molecules may be utilized in the subject methods and compositions as target molecules, including specific binding pairs, ligand and capture agent, biotin and streptavidin, antibody conjugates, and the like.

In some instances, the target molecule is a protein. Proteins of interest include, but are not limited to, those having a naturally-occurring amino acid sequence, fragments of naturally-occurring polypeptides, and non-naturally occurring polypeptides and fragments thereof. In some instances, the protein is a carrier protein. In some instances, the protein is an antibody. In some instances, the protein is an antibody fragment.

In certain embodiments, the target molecule is a hormone, a growth factor, a receptor, an enzyme, a cytokine, an osteoinductive factor, a colony stimulating factor or an immunoglobulin.

In certain embodiments, the target molecule may be one or more of the following: growth hormone, bovine growth hormone, insulin like growth factors, human growth hormone including n-methionyl human growth hormone, parathyroid hormone, thyroxine, insulin, proinsulin, amylin, relaxin, prorelaxin, glycoprotein hormones such as follicle stimulating hormone (FSH), leutinizing hormone (LH), hemapoietic growth factor, Her-2, fibroblast growth factor, prolactin, placental lactogen, tumor necrosis factors, mullerian inhibiting substance, mouse gonadotropin-associated polypeptide, inhibin, activin, vascular endothelial growth factors, integrin, nerve growth factors such as NGF-beta, insulin-like growth factor-I and II, erythropoietin, osteoinductive factors, interferons, colony stimulating factors, interleukins (e.g., a IL-4, IL-8, IL-1-a, IL-6, IL-12, IL-13, IL-17 or IL-23 protein), growth factor blockers (VEGF-A, -D, PDGF-B), a bi-specific blocker (e.g., VEGF-A+PDGF-B), a receptor agonist (e.g., Robo4), bone morphogenetic proteins, LIF, SCF, FLT-3 ligand, kit-ligand, SH3 domain, apoptosis protein, hepatocyte growth factor, hepatocyte growth factor receptor, neutravidin, maltose binding protein, angiostatin, aFGF, bFGF, TGF-alpha, TGF-beta, HGF, TNF-alpha, angiogenin, IL-8, thrombospondin, the 16-kilodalton N-terminal fragment of prolactin and endostatin.

In certain embodiments, the target molecule may be a therapeutic target protein such as, but not limited to: Raf kinase (a target for the treatment of melanoma), Rho kinase (a target in the prevention of pathogenesis of cardiovascular disease), nuclear factor kappaB (NF-.kappa.B, a target for the treatment of multiple myeloma), vascular endothelial growth factor (VEGF) receptor kinase (a target for action of anti-angiogenetic drugs), Janus kinase 3 (JAK-3, a target for the treatment of rheumatoid arthritis), cyclin dependent kinase (CDK) 2 (CDK2, a target for prevention of stroke), FMS-like tyrosine kinase (FLT) 3 (FLT-3; a target for the treatment of acute myelogenous leukemia (AML)), epidermal growth factor receptor (EGFR) kinase (a target for the treatment of cancer), protein kinase A (PKA, a therapeutic target in the prevention of cardiovascular disease), p21-activated kinase (a target for the treatment of breast cancer), mitogen-activated protein kinase (MAPK, a target for the treatment of cancer and arthritis), c-Jun NH.sub.2-terminal kinase (JNK, a target for treatment of diabetes), AMP-activated kinase (AMPK, a target for prevention and treatment of insulin resistance), lck kinase (a target for immuno-suppression), phosphodiesterase PDE4 (a target in treatment of inflammatory diseases such as rheumatoid arthritis and asthma), Abl kinase (a target in treatment of chronic myeloid leukemia (CML)), phosphodiesterase PDE5 (a target in treatment of erectile dysfunction), a disintegrin and metalloproteinase 33 (ADAM33, a target for the treatment of asthma), human immunodeficiency virus (HIV)-1 protease and HIV integrase (targets for the treatment of HIV infection), respiratory syncytial virus (RSV) integrase (a target for the treatment of infection with RSV), X-linked inhibitor of apoptosis (XIAP, a target for the treatment of neurodegenerative disease and ischemic injury), thrombin (a therapeutic target in the treatment and prevention of thromboembolic disorders), tissue type plasminogen activator (a target in prevention of neuronal death after injury of central nervous system), matrix metalloproteinases (targets of anti-cancer agents preventing angiogenesis), beta secretase (a target for the treatment of Alzheimer's disease), src kinase (a target for the treatment of cancer), fyn kinase, lyn kinase, zeta-chain associated protein 70 (ZAP-70) protein tyrosine kinase, extracellular signal-regulated kinase 1 (ERK-1), p38 MAPK, CDK4, CDK5, glycogen synthase kinase 3 (GSK-3), KIT tyrosine kinase, FLT-1, FLT-4, kinase insert domain-containing receptor (KDR) kinase, and cancer osaka thyroid (COT) kinase.

In certain embodiments, the target molecule is a target protein that is selected from the group consisting of a VEGF protein (e.g., VEGF-A, VEGF-C or VEGF-D), a RANKL protein, a NGF protein, a TNF-alpha protein, a SH2 domain containing protein, a SH3 domain containing protein, an IgE protein a BLyS protein (Oren et al., “Structural basis of BLyS receptor recognition”, Nature Structural Biology 9, 288-292, 2002), a PCSK9 protein (Ni et al., “A proprotein convertase subtilisin-like/kexin type 9 (PCSK9) C-terminal domain antibody antigen-binding fragment inhibits PCSK9 internalization and restores low density lipoprotein uptake”, J. Biol. Chem. 2010 Apr. 23; 285(17):12882-91), a DLL4 protein (Garber, “Targeting Vessel Abnormalization in Cancer”, JNCI Journal of the National Cancer Institute 2007 99(17):1284-1285), an Ang2 (Angiopoietin-2) protein, a Clostridium difficile Toxin A or B protein (e.g., Ho et al., “Crystal structure of receptor-binding C-terminal repeats from Clostridium difficile toxin A”, (2005) Proc. Natl. Acad. Sci. Usa 102: 18373-18378), a CTLA4 protein (Cytotoxic T-Lymphocyte Antigen 4), and fragments thereof. In certain embodiments, the target protein is a VEGF protein. In certain embodiments, the target protein is a SH2 domain containing protein (e.g., a 3BP2 protein) or a SH3 domain containing protein (e.g., a ABL or a Src protein).

In some instances, the target protein is selected from PDGF-B, Robo4, Htra1, hemagglutinin, Nav1.7, CD5, CD19, CD38, CD40, IGF-1R, GM-CSF, PCSK9, BlyS, Ang2, EGFR, HER2, Robo4, Htra1, CXCL5, Sclerostin, R-Spondin, MD-2, an Influenza HA hemagglutinin protein or a coiled coil mimic thereof, HCV, an HIV protein.

In some cases, the target molecule is a detectable label. A variety of detectable labels may be utilized in the subject methods, conjugates and compositions of the present disclosure. Examples of detectable labels include, but are not limited to, fluorescent molecules (e.g., autofluorescent molecules, molecules that fluoresce upon contact with a reagent, etc.), radioactive labels (e.g., ¹¹¹In, ¹²⁵I, ¹³¹I, ²¹²B, ⁹⁰Y, ¹⁸⁶Rh, and the like), biotin (e.g., to be detected through reaction of biotin and avidin), fluorescent tags, imaging reagents, and the like. Detectable labels also include peptides or polypeptides that can be detected by antibody binding, e.g., by binding of a detectably labeled antibody or by detection of bound antibody through a sandwich-type assay. Further examples of detectable labels include, but are not limited to, dye labels (e.g., chromophores, fluorophores, such as, but not limited to, Alexa Fluor® fluorescent dyes (e.g., Alexa Fluor® 350, 405, 430, 488, 532, 546, 555, 568, 594, 595, 610, 633, 635, 647, 660, 680, 700, 750, 790, and the like), coumarins, rhodamines (5-carboxyrhodamine and sulfo derivates thereof, e.g., 5-carboxy-disulfo-rhodamine, carbopyranins and oxazines, such as ATTO dyes (e.g., ATTO 390, 425, 465, 488, 495, 520, 532, 550, 565, 590, 594, 610, 611X, 620, 633, 635, 637, 647, 647N, 655, 665, 680, 700, 725 or 740), biophysical probes (spin labels, nuclear magnetic resonance (NMR) probes), Förster Resonance Energy Transfer (FRET)-type labels (e.g., at least one member of a FRET pair, including at least one member of a fluorophore/quencher pair), Bioluminescence Resonance Energy Transfer (BRET)-type labels (e.g., at least one member of a BRET pair), immunodetectable tags (e.g., FLAG, His(6), and the like), localization tags (e.g., to identify association of a tagged polypeptide at the tissue or molecular cell level (e.g., association with a tissue type, or particular cell membrane), and the like.

### Amine N-Oxide Linkers

As described above, the N-oxide functional group may be attached to a target molecule of interest indirectly via a linker that includes a reactive functional group capable of conjugation to a compatible functional group on the target molecule.

In some instances, the amine N-oxide linker is described by the formula:

Z¹—N(→O)(R⁴)-L-Z²

where Z¹ is a first molecule of interest, L is a linker and Z² is a functional group capable of conjugation to a second molecule of interest; and R⁴ is as defined above.

A variety of reactive functional groups may be utilized to attach an amine N-oxide linker to the target molecule. Any convenient methods and functional groups that find use in bioorthogonal or chemoselective conjugation reactions may be adapted for use in the subject methods to label a target molecule with an amine N-oxide, e.g., via chemoselective reaction with the amine N-oxide linker. Reactive functional groups of interest which may find use in the subject methods and compositions, include but are not limited to, aldehydes, azides, nitrones, nitrile oxides, diazo compounds, tetrazines, tetrazoles, quadrocyclanes, alkenes, alkynes (e.g., strained alkynes), iodobenzenes, alkylchlorides, active esters (e.g., NHS or sulfo-NHS ester), maleimide, thiols, amines, hydroxylamine, hydazide, hydrazine, epoxides, etc. Bioorthogonal ligation reactions of interest include, but are not limited to, those reactions described in Table 1 of Debets et al. “Bioorthogonal labelling of biomolecules: new functional handles and ligation methods”, Org. Biomol. Chem., 2013, 11, 6439-6455, the disclosure of which is herein incorporated by reference.

In some cases, the linker may include a self-immolative linker group. As used herein, the term “self-immolative” refers to a linker group that is capable of being cleaved by an adjacent triggering group. In certain embodiments, the amine N-oxide-linker comprises a group selected from the group consisting of para-amino-benzyloxycarbonyl (PABC), meta-amino-benzyloxycarbonyl (MABC), of ortho-amino-benzyloxycarbonyl (PABC), para-amino-benzyloxy (PABO), meta-amino-benzyloxy (MABO) ortho-aminobenzyl and para-aminobenzyl, where the amino group is converted to a stable amine-N-oxide using the methods described herein. In the subject methods, the stable amine N-oxide linker can be chemoselectively modified with a boron agent into a self-immolative linker capable of cleavage in vitro or in vivo. Thus, the subject methods may be utilized to provide for bioorthogonal cleavage, e.g., of a target molecule conjugate (e.g., as described herein).

### Target Molecule Conjugates

Aspects of the present disclosure include target molecule conjugates comprising a first target molecule and a second target molecule, covalently linked via an amine N-oxide-linker. The first and second target molecules may be independently selected from a biomolecule and a chemical entity (e.g., a drug or a detectable label). In some cases, the first and second target molecules are each independently a biomolecule. In certain cases, the first target molecule is a biomolecule and the second target molecule is a chemical entity.

In some instances, the conjugate is described by the formula:

Z¹—N(→O)(R⁴)-L-Z²

where Z¹ is a first target molecule of interest, L is an optional linker; Z² is a second target molecule of interest; and R⁴ is as defined above.

In some cases, the conjugate is modified according to the subject methods to produce a conjugate having a cleavable linker. The cleavable linker can be in some cases be spontaneously cleavable or cleavable by application of a stimulus, e.g., a pH condition or an enzyme. In certain instances, the conjugate is modified according to the subject methods to produce a conjugate where the linker remains non-cleavable. In such cases, the modified conjugate may be activated to provide for a desirable spectroscopic or biological property (e.g., as described herein).

In some embodiments, the conjugate is a polypeptide conjugate, e.g., a conjugate of a polypeptide and a second target molecule of interest. The polypeptides can be subjected to conjugation to provide for attachment of a wide variety of moieties via an amine N-oxide-linker (e.g., as described herein). Examples of moieties of interest include, but are not limited to, a drug, a detectable label, a small molecule, a water-soluble polymer, a peptide, and the like (also referred to a “payload” or “cargo” herein). Thus, the present disclosure provides a polypeptide conjugate as described above.

The moiety of interest may be provided as a component of a reactive partner (e.g., an amine N-oxide-linker) for reaction with a residue of a polypeptide. In certain embodiments, the methods of polypeptide conjugation are compatible with reaction conditions suitable for the polypeptide. For example, the reaction conditions may include a reaction mixture that includes water. In some cases, the reaction mixture may have a pH compatible with the polypeptide, such as, but not limited to, a pH of 4 to 11, or a pH of 5 to 10, or a pH of 6 to 9, or a pH of 6 to 8. In certain instances, the reaction mixture has a pH of 7. In some embodiments, the reaction conditions are performed at a temperature compatible with the polypeptide.

Provided the present disclosure, the ordinarily skilled artisan can readily adapt any of a variety of moieties to provide a reactive partner for conjugation to a polypeptide as contemplated herein. The ordinarily skilled artisan will appreciate that factors such as pH and steric hindrance (i.e., the accessibility of the modified amino acid residue to reaction with a reactive partner of interest) are of importance. Modifying reaction conditions to provide for optimal conjugation conditions is well within the skill of the ordinary artisan, and is routine in the art. Where conjugation is conducted with a polypeptide present in or on a living cell, the conditions are selected so as to be physiologically compatible. For example, the pH can be dropped temporarily for a time sufficient to allow for the reaction to occur but within a period tolerated by the cell (e.g., from about 30 min to 1 hour). Physiological conditions for conducting modification of polypeptides on a cell surface can be similar to those used in a ketone-azide reaction in modification of cells bearing cell-surface azides (see, e.g., U.S. Pat. No. 6,570,040).

In certain embodiments, the present disclosure provides a polypeptide conjugate, where the polypeptide is an antibody. As such, embodiments include an antibody conjugated to a moiety of interest via an amine N-oxide-linker, where an antibody conjugated to a moiety of interest is referred to as an “antibody conjugate.” An Ig polypeptide generally includes at least an Ig heavy chain constant region or an Ig light chain constant region, and can further include an Ig variable region (e.g., a V_{L} region and/or a V_{H} region). Ig heavy chain constant regions include Ig constant regions of any heavy chain isotype, non-naturally occurring Ig heavy chain constant regions (including consensus Ig heavy chain constant regions). An Ig constant region can be modified to be conjugated to a moiety of interest, where the moiety of interest is present in or adjacent a solvent-accessible loop region of the Ig constant region.

In some cases, an antibody conjugate of the present disclosure can include: 1) Ig heavy chain constant region conjugated to one or more moieties of interest, and an Ig light chain constant region conjugated to one or more moieties of interest; 2) an Ig heavy chain constant region conjugated to one or more moieties of interest, and an Ig light chain constant region that is not conjugated to a moiety of interest; or 3) an Ig heavy chain constant region that is not conjugated to a moiety of interest, and an Ig light chain constant region conjugated to one or more moieties of interest. A subject antibody conjugate can also include variable VH and/or VL domains. As described above, the one or more moieties of interest may be conjugated to the Ig heavy chain constant region or the Ig light chain constant region at a single amino acid residue (e.g., one or two moieties of interest conjugated to a single amino acid residue), or conjugated to the Ig heavy chain constant region and/or the Ig light chain constant region at two or more different amino acid residues.

An antibody conjugate of the present disclosure can include, as the conjugated moiety, any of a variety of compounds, as described herein, e.g., a drug (e.g., a peptide drug, a small molecule drug, and the like), a water-soluble polymer, a detectable label, a synthetic peptide, etc.

An antibody conjugate can have any of a variety of antigen-binding specificities, as described above, including, e.g., an antigen present on a cancer cell; an antigen present on an autoimmune cell; an antigen present on a pathogenic microorganism; an antigen present on a virus-infected cell (e.g., a human immunodeficiency virus-infected cell), e.g., CD4 or gp120; an antigen present on a diseased cell; and the like. For example, an antibody conjugate can bind an antigen, as noted above, where the antigen is present on the surface of the cell. An antibody conjugate of the present disclosure can bind antigen with a suitable binding affinity, e.g., from 5×10⁻⁶ M to 10⁻⁷ M, from 10⁻⁷ M to 5×10⁷ M, from 5×10⁻⁷ M to 10⁻⁸ M, from 10⁻⁸ M to 5×10⁻⁸ M, from 5×10⁻⁸ M to 10⁻⁹ M, or a binding affinity greater than 10⁻⁹ M.

As non-limiting examples, a subject antibody conjugate can bind an antigen present on a cancer cell (e.g., a tumor-specific antigen; an antigen that is over-expressed on a cancer cell; etc.), and the conjugated moiety can be a cytotoxic compound (e.g., a cytotoxic small molecule, a cytotoxic synthetic peptide, etc.). For example, a subject antibody conjugate can be specific for an antigen on a cancer cell, where the conjugated moiety is a cytotoxic compound (e.g., a cytotoxic small molecule, a cytotoxic synthetic peptide, etc.).

As further non-limiting examples, a subject antibody conjugate can bind an antigen present on a cell infected with a virus (e.g., where the antigen is encoded by the virus; where the antigen is expressed on a cell type that is infected by a virus; etc.), and the conjugated moiety can be a viral fusion inhibitor. For example, a subject antibody conjugate can bind an antigen present on a cell infected with a virus, and the conjugated moiety can be a viral fusion inhibitor.

Embodiments of the present disclosure also include polypeptide conjugates where the polypeptide is a carrier protein. For example, carrier proteins can be covalently and site-specifically bound to drug to provide a drug-containing scaffold. A carrier protein can be site-specifically conjugated to a covalently bound molecule of interest, such as a drug (e.g., a peptide, a small molecule drug, and the like), detectable label, etc. In certain embodiments, drug-scaffold conjugates can provide for enhanced serum half-life of the drug.

In general a “carrier protein” is a protein that is biologically inert, is susceptible to modification as disclosed herein, and which can provide for solvent-accessible presentation of the moiety of interest conjugated to the carrier protein through a modified amino acid residue in the carrier protein (e.g., through an oxime or hydrazone bond within the converted sulfatase motif of an aldehyde tagged carrier protein) in a physiological environment. “Biologically inert” is meant to indicate the carrier protein exhibits clinically insignificant or no detectable biological activity when administered to the appropriate subject, such as when administered to a human subject. Thus, carrier proteins are biologically inert in that they, for example, are of low immunogenicity, do not exhibit significant or detectable targeting properties (e.g., do not exhibit significant or detectable activity in binding to a specific receptor), and exhibit little or no detectable biological activity that may interfere with activity of the moiety (e.g., drug or detectable label) conjugated to the aldehyde-tagged carrier protein. By “low immunogenicity” is meant that the carrier protein elicits little or no detectable immune response upon administration to a subject, such as a mammalian subject, e.g., a human subject. Carrier proteins can be provided in monomeric or multimeric (e.g., dimeric) forms.

Carrier proteins having a three-dimensional structure when folded that provides for multiple different solvent-accessible sites that are amenable to modification (and thus conjugation to a moiety of interest) are of interest. In general, carrier proteins of interest are those that are of a size and three-dimensional folded structure so as to provide for presentation of the conjugated moiety of interest on solvent accessible surfaces in a manner that is sufficiently spatially separated so as to provide for activity and bioavailability of the conjugated moiety or moieties of interest. The carrier protein may be selected according to a variety of factors including, but not limited to, the moiety (e.g., drug or detectable label) to be conjugated to the carrier protein.

Accordingly, any of a wide variety of polypeptides can be suitable for use as carrier proteins for use in the carrier protein conjugates of the present disclosure. Such carrier proteins can include those having a naturally-occurring amino acid sequence, fragments of naturally-occurring polypeptides, and non-naturally occurring polypeptides and fragments thereof.

Examples of carrier proteins include, but are not limited to, albumin and fragments thereof (e.g., human serum albumin, bovine serum albumin, and the like), transferrin and fragments thereof (e.g. human transferrin), and Fc fragments having reduced binding to a mammalian Fc receptor, particularly a human Fc receptor (e.g., a modified Fc fragment of an antibody (e.g., IgG), such as a mammalian antibody, e.g., a human antibody). Examples of modified Fc fragments having reduced Fc receptor binding are exemplified by the Fc fragments of Herceptin (trastuzumab) and Rituxan (Rituximab), which contain point mutations that provide for reduced Fc receptor binding (see, e.g., Clynes et al., Nature Medicine (2000), 6, 443-446). Alternatively or in addition, the isotype of the Fc fragment can be selected according to a desired level of Fc receptor binding (e.g., use of an Fc fragment of an IgG4 isotype human heavy chain constant region rather than from IgG1 or IgG3. (see, e.g., Fridman FASEB J 1991 September; 5 (12): 2684-90). In general, carrier proteins can be at least about 4 kDa (e.g., about 50 amino acid residues in length), usually at least about 25 kDa, and can be larger in size (e.g., transferrin has a molecular weight of 90 kDa while Fc fragments can have molecular weights of 30 kDa to 50 kDa).

The conjugates described herein can be used for a variety of applications including, but not limited to, visualization using fluorescence or epitope labeling (e.g., electron microscopy using gold particles equipped with reactive groups for conjugation to the compounds and conjugates described herein); protein immobilization (e.g., protein microarray production); protein dynamics and localization studies and applications; and conjugation of proteins with a moiety of interest (e.g., moieties that improve a parent protein's half-life (e.g., poly(ethylene glycol)), targeting moieties (e.g., to enhance delivery to a site of action), and biologically active moieties (e.g., a therapeutic moiety).

The polypeptide conjugate may include a polypeptide conjugated to a moiety or moieties that provide for one or more of a wide variety of functions or features. In general, examples of moieties include, but are not limited to, the following: detectable labels (e.g., fluorescent labels); light-activated dynamic moieties (e.g., azobenzene mediated pore closing, azobenzene mediated structural changes, photodecaging recognition motifs); water soluble polymers (e.g., PEGylation); purification tags (e.g., to facilitate isolation by affinity chromatography (e.g., attachment of a FLAG epitope); membrane localization domains (e.g., lipids or glycophosphatidylinositol (GPI)-type anchors); immobilization tags (e.g., to facilitate attachment of the polypeptide to a surface, including selective attachment); drugs (e.g., to facilitate drug targeting, e.g., through attachment of the drug to an antibody); targeted delivery moieties, (e.g., ligands for binding to a target receptor (e.g., to facilitate viral attachment, attachment of a targeting protein present on a liposome, etc.)), and the like.

Specific, non-limiting examples are provided below.

### Drugs for Conjugation to a Polypeptide

Any of a number of drugs are suitable for use, or can be modified to be rendered suitable for use, as a reactive partner to conjugate to a polypeptide. Examples of drugs include small molecule drugs and peptide drugs. Thus, the present disclosure provides drug-polypeptide conjugates.

“Small molecule drug” as used herein refers to a compound, e.g., an organic compound, which exhibits a pharmaceutical activity of interest and which is generally of a molecular weight of 800 Da or less, or 2000 Da or less, but can encompass molecules of up to 5 kDa and can be as large as 10 kDa. A small inorganic molecule refers to a molecule containing no carbon atoms, while a small organic molecule refers to a compound containing at least one carbon atom.

“Peptide drug” as used herein refers to amino-acid containing polymeric compounds, and is meant to encompass naturally-occurring and non-naturally-occurring peptides, oligopeptides, cyclic peptides, polypeptides, and proteins, as well as peptide mimetics. The peptide drugs may be obtained by chemical synthesis or be produced from a genetically encoded source (e.g., recombinant source). Peptide drugs can range in molecular weight, and can be from 200 Da to 10 kDa or greater in molecular weight.

In some cases, the drug is a cancer chemotherapeutic agent. For example, where the polypeptide is an antibody (or fragment thereof) that has specificity for a tumor cell, the antibody can be modified as described herein to include a modified amino acid, which can be subsequently conjugated to a cancer chemotherapeutic agent. Cancer chemotherapeutic agents include non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones. Peptidic compounds can also be used.

Suitable cancer chemotherapeutic agents include dolastatin and active analogs and derivatives thereof; and auristatin and active analogs and derivatives thereof (e.g., Monomethyl auristatin D (MMAD), monomethyl auristatin E (MMAE), monomethyl auristatin F (MMAF), and the like). See, e.g., WO 96/33212, WO 96/14856, and U.S. Pat. No. 6,323,315. For example, dolastatin 10 or auristatin PE can be included in an antibody-drug conjugate of the present disclosure. Suitable cancer chemotherapeutic agents also include maytansinoids and active analogs and derivatives thereof (see, e.g., EP 1391213; and Liu et al (1996) Proc. Natl. Acad. Sci. USA 93:8618-8623); duocarmycins and active analogs and derivatives thereof (e.g., including the synthetic analogues, KW-2189 and CB 1-TM1); and benzodiazepines and active analogs and derivatives thereof (e.g., pyrrolobenzodiazepine (PBD).

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation; therefore compounds that bind to the estrogen receptor are used to block this activity. Corticosteroids may inhibit T cell proliferation.

Other suitable chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc. Other anti-proliferative agents of interest include immunosuppressants, e.g. mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

Taxanes are suitable for use. “Taxanes” include paclitaxel, as well as any active taxane derivative or pro-drug. “Paclitaxel” (which should be understood herein to include analogues, formulations, and derivatives such as, for example, docetaxel, TAXOL™, TAXOTERE™ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis).

Paclitaxel should be understood to refer to not only the common chemically available form of paclitaxel, but analogs and derivatives (e.g., Taxotere™ docetaxel, as noted above) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

Biological response modifiers suitable for use include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) IFN-α; (7) IFN-γ; (8) colony-stimulating factors; and (9) inhibitors of angiogenesis.

### Methods of Treatment

The polypeptide-drug conjugates of the present disclosure find use in conjunction with boron agents in treatment of a condition or disease in a subject that is amenable to treatment by administration of the parent drug (i.e., the drug prior to conjugation to the polypeptide). By “treatment” is meant that at least an amelioration of the symptoms associated with the condition afflicting the host is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g., prevented from happening, or stopped, e.g. terminated, such that the host no longer suffers from the condition, or at least the symptoms that characterize the condition. Thus treatment includes: (i) prevention, that is, reducing the risk of development of clinical symptoms, including causing the clinical symptoms not to develop, e.g., preventing disease progression to a harmful state; (ii) inhibition, that is, arresting the development or further development of clinical symptoms, e.g., mitigating or completely inhibiting an active disease; and/or (iii) relief, that is, causing the regression of clinical symptoms.

In the context of cancer, the term “treating” includes any or all of: reducing growth of a solid tumor, inhibiting replication of cancer cells, reducing overall tumor burden, and ameliorating one or more symptoms associated with a cancer.

The subject to be treated can be one that is in need of therapy, where the host to be treated is one amenable to treatment using the parent drug. Accordingly, a variety of subjects may be amenable to treatment using the polypeptide-drug conjugates disclosed herein. Generally, such subjects are “mammals”, with humans being of interest. Other subjects can include domestic pets (e.g., dogs and cats), livestock (e.g., cows, pigs, goats, horses, and the like), rodents (e.g., mice, guinea pigs, and rats, e.g., as in animal models of disease), as well as non-human primates (e.g., chimpanzees, and monkeys).

In some embodiments, the method includes administering a polypeptide-drug conjugate in conjunction with a boron agent. By “in combination with” or “in conjunction with”, is meant that an amount of the conjugate is administered anywhere from simultaneously to up to 5 hours or more, e.g., 10 hours, 15 hours, 20 hours or more, prior to, the boron agent. In certain embodiments, the boron agent and conjugate are administered sequentially, e.g., where the boron agent is administered after the conjugate. In yet other embodiments, the boron agent and metabolizing enzyme inhibitor are administered simultaneously, e.g., where the boron agent and conjugate are administered at the same time as two separate formulations, or are combined into a single composition, that is administered to the subject. Regardless of whether the boron agent and conjugate are administered sequentially or simultaneously, as illustrated above, or any effective variation thereof, the agents are considered to be administered together or in combination for purposes of the present invention. Routes of administration of the two agents may vary.

The amount of polypeptide-drug conjugate administered can be initially determined based on guidance of a dose and/or dosage regimen of the parent drug. Similarly, the amount of boron agent administered can be initially determined based on the amount effective to release a dose and/or dosage regimen of the parent drug in the conjugate. In general, the polypeptide-drug conjugates can provide for targeted delivery and/or enhanced serum half-life of the bound drug, thus providing for at least one of reduced dose or reduced administrations in a dosage regimen. Thus, the polypeptide-drug conjugates can provide for reduced dose and/or reduced administration in a dosage regimen relative to the parent (unconjugated) drug prior to being conjugated in a polypeptide-drug conjugate of the present disclosure.

Furthermore, as noted above, because the polypeptide-drug conjugates can provide for controlled stoichiometry of drug delivery, dosages of polypeptide-drug conjugates can be calculated based on the number of drug molecules provided on a per polypeptide-drug conjugate basis. In some embodiments, multiple doses of a polypeptide-drug conjugate are administered. The frequency of administration of a polypeptide-drug conjugate can vary depending on any of a variety of factors, e.g., severity of the symptoms, condition of the subject, etc. For example, in some embodiments, a polypeptide-drug conjugate is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

### Utility

The subject methods and compositions find use in a variety of applications, including research applications, therapeutic applications and diagnostic applications.

Research applications of interest include any application where the selective manipulation of molecules, cells, particles and surfaces is of interest, including the manipulation, tagging and tracking of biomolecules (e.g., proteins) in vitro and in vivo.

The subject methods and compositions also find use in diagnostic applications, e.g., in tumor-pretargeted radioimmunoimaging, where a bioorthogonal cleavage reaction can deliver a radiolabel to a site of interest in vivo.

Therapeutic applications of interest include applications where antibody-drug-conjugates (ADC) find use, where the linker between the tumor-bound antibody and the drug is selectively cleaved in vivo through reaction with a boron agent, which is administered in a second step. For example, Versteegen et al (Angew. Chem. Int. Ed. 2013, 52, 14112) describe a tetrazine-based bioorthogonal cleavage to release doxorubicin from an ADC. This approach does not rely on the currently employed endogenous intracellular ADC activation mechanisms (such as enzymatic activation), and thus expands the scope of suitable ADC.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Demonstration of a Bioorthogonal N-Oxide/Boron Agent Chemistry

An application of interest of bioorthogonal chemistry is related to modulation of the activity of a target molecule under the control of a small molecule switch. To this end, reactions that involve bioorthogonal bond cleavage, rather than formation, are of interest. Organism-specific variations in biological reactivity space point to bioorthogonal reaction partners not just from outside but also within biology. Natural organisms possess considerable metabolic diversity, such that functional groups endogenous to one species (e.g., terminal alkynes) can be orthogonal in another.

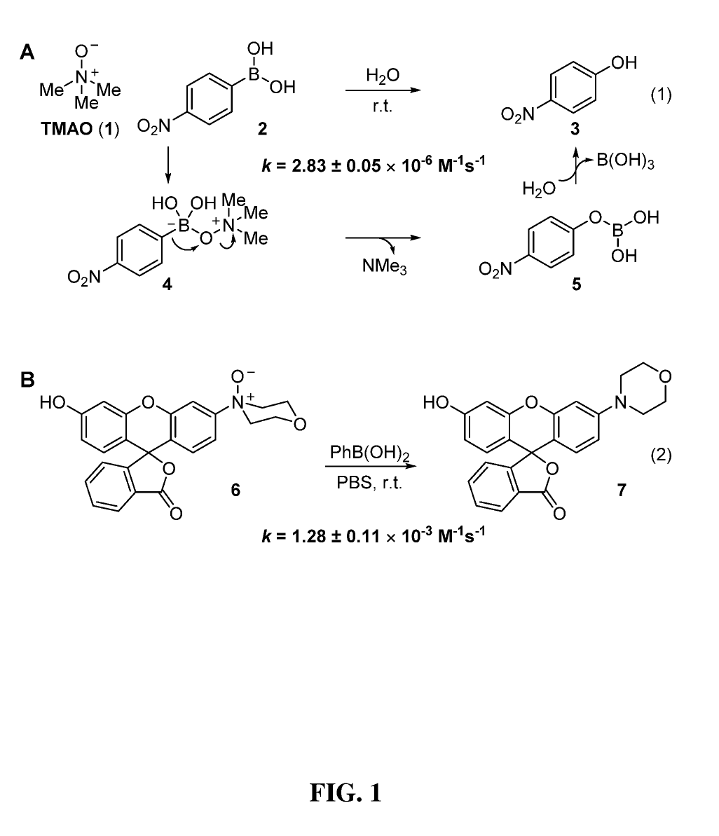

Trimethylamine N-oxide (TMAO, 1, FIG. 1) provides another example. Elasmobranchs (see, e.g., Yancey et al., Science 1982, 217, 1214) deep-sea teleosts (see e.g., Yancey et al., Proc. Natl. Acad. Sci. 2014, 111, 4461), and a variety of osmoconforming marine invertebrates acquire high concentrations of urea to maintain high internal osmolalities. Chaotropic effects of urea are countered by proportionally high concentrations of the potent kosmotrope TMAO (see e.g., Yancey, P. H. Amer. Zool. 2001, 41, 699). In deep-sea fish where the kosmotropic effects of TMAO are essential in countering immense hydrostatic pressures, concentrations of TMAO can be as high as 400 mM (see e.g., Yancey et al., Proc. Natl. Acad. Sci. 2014, 111, 4461). Even at very high concentrations, N-oxides are unreactive toward biomolecules. Most other organisms, including humans, do not use TMAO as a component of osmoregulation. They consequently possess very low concentrations of this analyte, which is present as a metabolic byproduct of trimethylamine generated by gut microbiota. N-oxides were identified as biocompatible reaction partners.

Similarly, boron-containing compounds fit the description that they are sometimes, yet infrequently, found in biological systems. For example, natural product antibiotics such as boromycin and aplasmomycin incorporate boron into their structures. Additionally, boronic acids have a relatively benign toxicology profile (see e.g., Trippier et al., MedChemComm 2010, 1, 183) and have been incorporated into chemical biology tools such as the fluorogenic tetraserine-binding and peroxide-responsive probes. Given the positive qualities of this functional group, N-oxides were combined with boronic acids to generate a new bioorthogonal reaction.

Hydroxydeboration reactions between TMAO and alkyl boranes (see e.g., Koster, R.; Morita, Y. Liebigs Ann. Chem. 1967, 704, 70; Kabalka, G. W.; Hedgecock, H. C. J. Org. Chem. 1975, 40, 1776) can have high functional group tolerance and quantitative yields to render it a mild, reliable alternative to hydrogen peroxide-mediated deborylative oxidations in total organic syntheses.

A first experiment was performed to determine the kinetic parameters for the reaction between TMAO (1) and p-nitrophenylboronic acid (2) in water (see FIG. 1, panel A). Reaction progress was monitored by UV/vis absorption of product p-nitrophenol (3) under pseudo-first order conditions. A second-order rate constant of 2.83±0.05×10⁻⁶ M⁻¹s⁻¹ was determined for the reaction at room temperature, several orders of magnitude below current standards of bioorthogonal reactivity (see e.g., Lang et al., ACS Chem. Biol. 2014, 9, 16; McKay et al. Chem. Biol. 2014, 21, 1075; Patterson et al., ACS Chem. Biol. 2014, 9, 592; (e) Sletten et al., Angew. Chem. Int. Ed. 2009, 48, 6974) but nonetheless a starting point for kinetic optimization.

In confirming that the concomitant C—B bond migration and N—O bond cleavage events are rate limiting (FIG. 1, panel A), it was expected that the principal determinants of reaction rate would be the leaving group ability of the tertiary amine nucleofuge and the migratory aptitude of the boronic acid. Focusing first on the former, the reaction could indeed be accelerated by turning to N,N-dialkylaryl N-oxides, which produce superior leaving groups compared to trialkylamines (see e.g., Zhu et al. Org. Lett. 2012, 14, 3494). Kinetic parameters for this reaction were measured by employing a fluorogenic N,N-dialkylaryl N-oxide 6 (see e.g., Niwa et al., Org. Biomol. Chem. 2014, 12, 6590; Hirayama et al., Chem. Sci. 2013, 4, 1250) obtained through mCPBA-mediated oxidation of the parent rhodol fluorophore (Peng, T.; Yang, D. Org. Lett. 2010, 12, 496). The reaction of N-oxide 6 with phenylboronic acid in phosphate-buffered saline (PBS, pH 7.4) proceeded with a second-order rate constant of 1.28±0.11×10⁻³ M−1s−1 (FIG. 1B), three orders of magnitude greater in rate acceleration relative to our baseline reaction using TMAO.

In developing even faster rates for use in biological systems, the migratory aptitude of the boronic acid was optimized. Improvements in rate could be achieved through direct weakening of the dissociating bond. Accordingly, the migrating C—B bond was exchanged for a B—B bond. The reduction of N-oxides by bis(pinacolato)diboron (B₂pin₂), (see e.g., Carter et al., Bifunctional Lewis Acid Reactivity of Diol-Derived Diboron Reagents. In Group 13 Chemistry: From Fundamentals to Applications, Shapiro, P. J.; Atwood, D. A., Eds. American Chemical Society: Washington, D.C., 2002; pp 70-87) exploits the cleavage of a weak B—B bond (68 kcal/mol) and formation of strong B—O bonds (125 kcal/mol) to provide an enthalpic driving force of ˜180 kcal/mol. This powerful reaction, which superficially effects nothing more than deoxygenation, has seen scant use in the chemical literature.

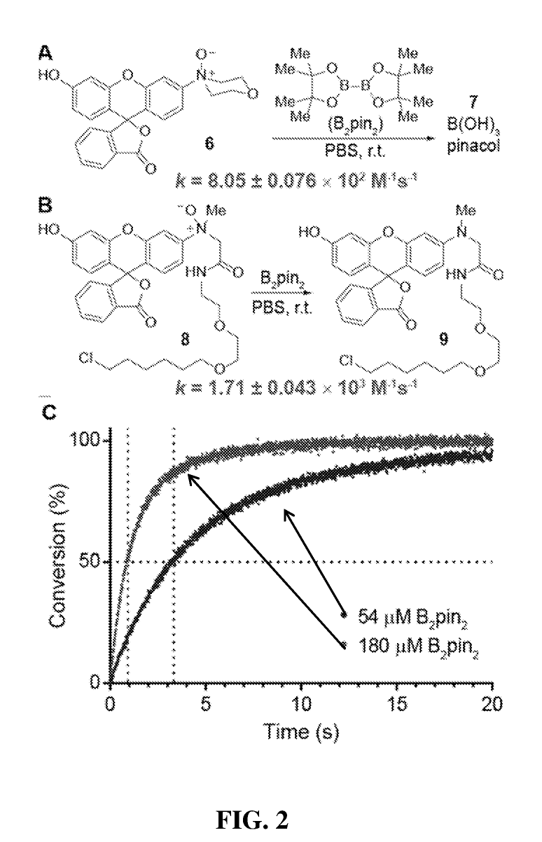

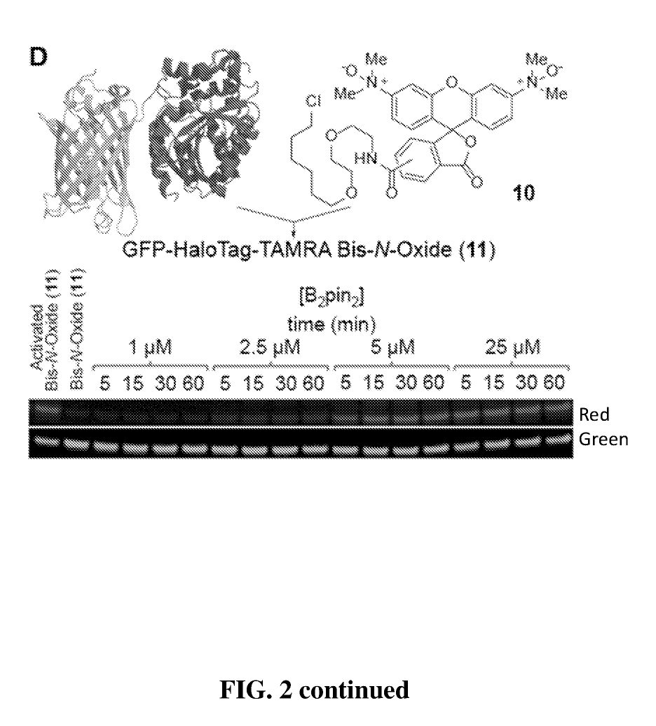

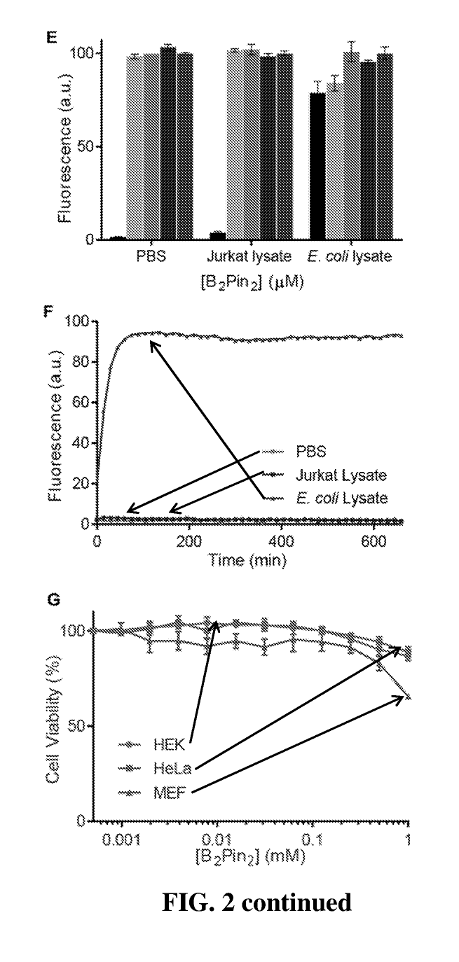

The kinetics of the reaction were first evaluated using N-oxide 6 (FIG. 2, panel A). Impressively, fluorescence measurements obtained on a stopped-flow fluorometer under pseudo-first order conditions revealed a second-order rate constant of 8.05±0.076×10² M⁻¹s⁻¹ in PBS (pH 7.4). A HaloTag linker-bound profluorophore 8 was synthesized, designed for use in cell labeling studies, and found the second-order rate constant for its reaction with B₂pin₂ to be even higher at 1.71±0.043×10³ M⁻¹s⁻¹, likely due to steric relaxation (FIG. 2, panel B).

The reaction was then performed on a biomolecule. The 34-kDa HaloTag protein was ligated to compound 8 to produce HaloTag-8, which was purified by size exclusion chromatography and treated with B₂pin₂ under pseudo-first-order conditions. Analysis by stopped-flow fluorometry revealed a second-order rate constant of 2.30±0.073×10³ M⁻¹s⁻¹ (FIG. 2, panel C). It should be noted, however, that pseudo-first-order kinetics performed under saturating conditions can obscure important information regarding the deactivation of the diboron reagent through sequestration or off-target reactivity. To address this issue, a 64-kDa GFP-HaloTag fusion protein was expressed, ligated to compound 10 to produce GFP-HaloTag-10. Conjugate 11 (500 nM) was then treated with stoichiometric to slightly superstoichiometric quantities of B₂pin₂ (1-25 μM) and analyzed by in-gel fluorescence imaging. FIG. 2, panel D shows that 5-10 equiv. B₂pin₂ are necessary to fully reduce conjugated fluorophore 10 in <15 min. Provided that 2 equiv. of reductant are theoretically required, this experiment validates the robustness of the reaction and suggests minimal off-target reactivity.

Having verified the compatibility of our N-oxide-diboron reaction with proteins, the viability of the reaction was explored in mammalian (Jurkat) and bacterial (E. coli) cell lysates (FIG. 2, panel E). Lysates were made to a final concentration of 1 mg/mL protein and variable concentrations of B₂pin₂ were reacted with 1 μM probe 6. Fluorescence intensities were then measured after 30 min. Consistent with our prior kinetic data, adding just 5 equiv. of B₂pin₂ was sufficient to drive the reaction to completion in mammalian cell lysate within the allotted time. Interestingly, the results were quite different in bacterial cell lysate as even the negative control displayed high levels of fluorescence in that system. This result suggests that N-oxides are unstable in E. coli lysate—a half-life of ˜15 min was measured—likely due to reduction by chemical or enzymatic processes (FIG. 2, panel F). No degradation was observed in Jurkat cell lysate.

In order to ascertain the toxicity of diboron reagents to cells, three mammalian cell lines (HEK293T, HeLa and MEF) were subjected to the MTT cell viability assay (FIG. 2G). Each cell line was treated with 2-fold serial dilutions of B₂pin₂ starting at 1 mM in 0.5% DMSO/DMEM. The maximum concentration of B₂pin₂ was dictated by its solubility limitations in the medium. The MTT assays indicated that the human cell lines are relatively insensitive to diboron, at least up to the concentrations that were tested, and while MEFs showed some toxicity at higher diboron levels, the IC₅₀ was >1 mM.

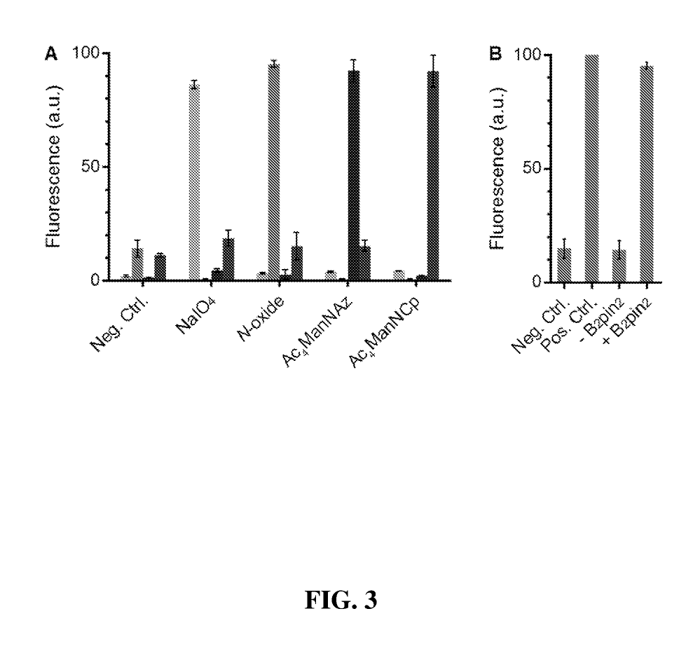

Next, the mutual orthogonality of the N-oxide-diboron reaction was demonstrated with a representative cohort of bioorthogonal reactions commonly in use today: the aminooxy-aldehyde condensation, the azide-cyclooctyne cycloaddition, and the tetrazine-cyclopropene ligation (FIG. 3). And to demonstrate the robustness of the new reaction, this experiment was executed amidst the complexity of a cellular system. Utilizing a combination of chemical, genetic, and metabolic engineering techniques, four populations of HEK293T cells were endowed with distinct cell surface modifications. Population 1 was modified by sodium periodate to display cell surface aldehydes derived from sialic acid moieties (see e.g., Zeng et al., Nat Meth 2009, 6, 207); population 2 was modified with azides through the metabolic incorporation of Ac₄ManNAz (see e.g., Saxon, E.; Bertozzi, C. R. Science 2000, 287, 2007); population 3 was modified with a cyclopropene through the metabolic incorporation of Ac4ManNCp (see e.g., Späte et al., Bioconjugate Chem. 2014, 25, 14); and population 4 was modified by cell surface expression of an N-terminal HaloTag-EGFR fusion protein followed by the enzymatic ligation of HaloTag-linked bis-N-oxide TAMRA profluorophore 10. Each cell population was labeled with a distinct combination of Hoechst 33342 and Syto 41 nuclear stains, combined, then treated with a reagent cocktail consisting of 10 mM aniline, 100 μM aminooxy-Alexa Fluor 488, 10 μM DIBAC-Cy5, 20 μM tetrazine-Cy7, and 100 μM B₂pin₂ in pH 6.7 PBS. The mélange of cells was analyzed by flow cytometry.

Each of the cell types was labeled highly selectively by its corresponding bioorthogonal partner, and the labeling efficiency was undiminished in the presence of the complete complement of reactive functional groups. Notably, aminooxy functional groups are fully compatible with diboron, and the N-oxide functional group is neither reduced by nor reactive toward tetrazines, which are known to be susceptible to degradation through nucleophilic attack.

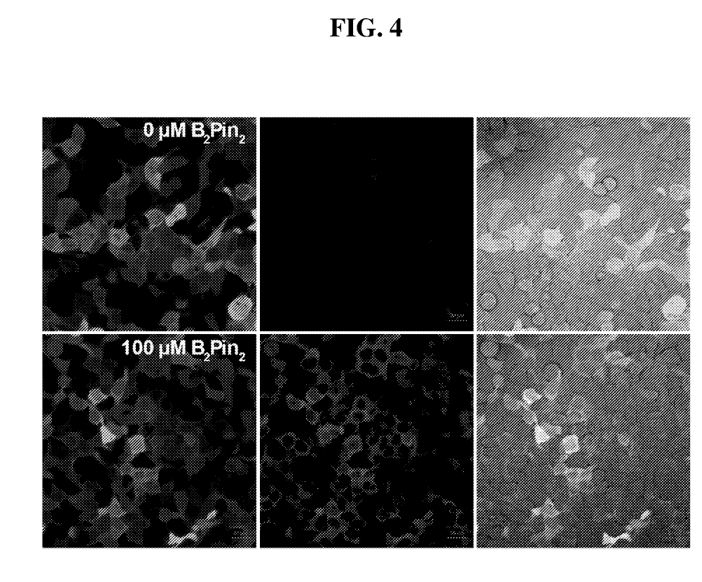

Finally, to demonstrate the utility of the N-oxide-diboron reaction in an intracellular uncaging application, the membrane permeability of each of the compounds was determined and their bioorthogonality in live cells evaluated. HEK293T cells were transiently transfected with a cytosolic GFP-HaloTag fusion construct, then treated with 100 M compound 10. After three washes, the cells were treated with 0 μM, 10 μM, 100 μM, or 1 mM B₂pin₂ in media for 45 min (FIG. 4, S1). The TAMRA signal increased upon addition of B₂pin₂ and also co-localized with the GFP signal. These data indicate that both profluorophore 10 and diboron are cell permeable, and the reaction can been performed intracellularly.

A powerful new reaction between N-oxides and boron reagents is demonstrated. The reaction features fast reaction kinetics that rival the fastest conventional bioorthogonal reactions while introducing little used functionality. The reaction conditions are benign toward live cells and it is intracellular compatibility.

### Example 2: Synthesis of N-Oxides and Boron Agents

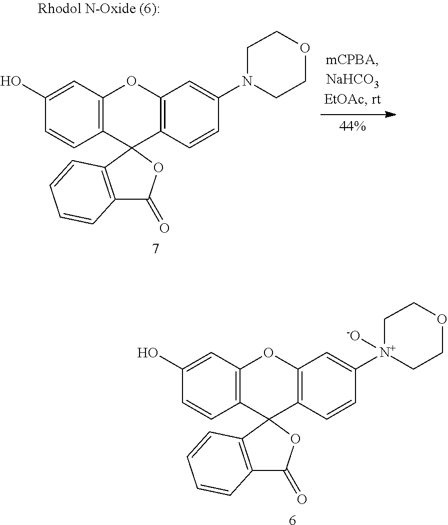

Trifluoroacetic acid (1 mL) was introduced via syringe to a solution of rhodol 7 (Peng, T.; Yang, D. Org. Lett. 2010, 12, 496-499) (14.3 mg, 32.1 μmol, 1 equiv) in dichloromethane (1 mL) at room temperature. After 1 h, the solution was concentrated to dryness under reduced pressure. The residue was dissolved in toluene (10 mL), then concentrated under reduced pressure. The resulting residue was dissolved in ethyl acetate (3 mL). Sodium bicarbonate (8.1 mg, 96.3 μmol, 3.00 equiv) and m-chloroperbenzoic acid (5.5 mg, 32.1 μmol, 1.00 equiv) were successively added to the solution at room temperature. After 1 h, the solution was concentrated to dryness. The pink residue was dissolved in methanol (4 mL), filtered, and purified by preparative HPLC on a C₁₈ column (20 mL/min, time (min), % MeCN in H₂O+0.1% TFA: 0, 0; 60, 100) to afford rhodol N-oxide 6 (5.9 mg, 14.1 μmol, 44%) as a pink film.

¹H NMR (500 MHz, CD₃OD) δ 8.11-8.04 (m, 2H), 7.78 (dt, J=24.5, 7.4 Hz, 2H), 7.70 (dd, J=9.0, 2.8 Hz, 1H), 7.23 (d, J=7.6 Hz, 1H), 7.16 (d, J=8.9 Hz, 1H), 7.23 (d, J=7.6 Hz, 1H), 7.16 (d, J=8.9 Hz, 1H), 6.78 (d, J=2.4 Hz, 1H), 6.68 (d, J=8.7 Hz, 1H), 6.63 (dd, J=8.7, 2.5 Hz, 1H), 4.46-4.33 (m, 4H), 4.12 (d, J=10.5 Hz, 2H), 3.98 (d, J=10.8 Hz, 2H). ¹³C NMR (126 MHz, CD₃OD) δ 170.7, 161.7, 153.9, 153.4, 153.2, 151.4, 137.0, 131.8, 131.7, 130.2, 127.5, 126.2, 125.1, 124.3, 116.3, 114.6, 111.2, 110.3, 103.6, 83.0, 67.0, 62.4. FTIR (thin film, cm⁻¹) 1765 (s), 1671 (m), 1612 (s), 1465 (w), 1429 (s), 1248 (w), 1193 (s), 1122 (s). HRMS (ESI, m/z) calculated for C₂₄H₂₀NO₆ [M⁺H]⁺: 418.1285; found: 418.1276.

### 5(6)-HaloTag Tetramethylrhodamine Bis-N-Oxide (10)

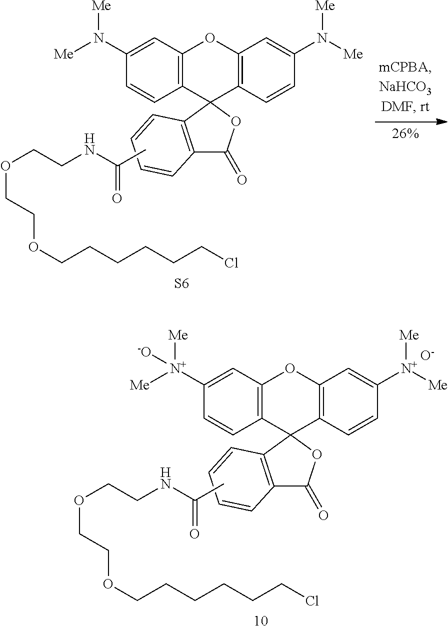

Sodium bicarbonate (139.9 mg, 1.66 mmol, 30.0 equiv) and m-chloroperbenzoic acid (191.6 mg, 1.11 mmol, 20.0 equiv) were added sequentially to a solution of 5(6)-HaloTag tetramethylrhodamine S6 (35.3 mg, 55.5 μmol, 1 equiv) in N,N-dimethylformamide (1.50 mL) at room temperature. After 30 m, the solution was cooled to −78° C., and N,N-diisopropylethylamine (241.6 μL, 1.39 mmol, 25.0 equiv) was added. The reaction mixture was then diluted with ice-water (10 mL), and immediately allowed to warm to room temperature over 30 min. The solution was purified by automated flash column chromatography on a C₁₈ column (Biotage SNAP KP-C18-HS, 30 g, 50 mL/min, time (min), % MeCN in H₂O: 0, 0; 4.5, 0; 18, 100). Column fractions containing tetramethylrhodamine bis-N-oxides 10 were collected and concentrated under reduced pressure. This mixture was purified by preparative HPLC on a C₁₈ column (20 mL/min, time (min), % MeCN in H₂O+0.1% TFA: 0, 0; 60, 100). The clean column fractions were collected and neutralized by the addition of sodium bicarbonate (200 mg) and concentrated under reduced pressure. The residue was dissolved in H₂O (10 mL) and desalted on a C₁₈ column (Biotage SNAP KP-C18-HS, 30 g, 50 mL/min; time (min), % MeCN in H₂O: 0, 0; 9, 100) to afford a 1.5:1 regioisomeric mixture of tetramethylrhodamine bis-N-oxides 10 (9.7 mg, 14.5 μmol, 26%) as a slightly pink film.

¹H NMR (500 MHz, CD₃OD) δ 8.56 (s, 0.6H), 8.27 (d, J=8.1 Hz, 0.6H), 8.23-8.12 (m, 2.8H), 7.77 (d, J=8.5 Hz, 2H), 7.68 (s, 0.4H), 7.39 (d, J=8.1 Hz, 0.6H), 7.35-7.23 (m, 2H), 4.00 (s, 12H), 3.75-3.40 (m, 12H), 1.81-1.66 (m, 2H), 1.64-1.49 (m, 2H), 1.49-1.31 (m, 4H). ¹³C NMR (126 MHz, CD₃OD) δ 169.1, 169.1, 167.8, 167.8, 155.6, 153.9, 152.4, 152.3, 152.1, 152.1, 143.1, 138.9, 136.4, 131.8, 131.7, 131.0, 128.7, 127.2, 127.0, 125.6, 125.3, 124.1, 122.9, 122.8, 117.3, 117.3, 111.1, 111.1, 80.8, 80.8, 72.2, 72.1, 71.3, 71.2, 71.2, 71.1, 70.4, 70.3, 61.0, 61.0, 45.7, 45.7, 41.2, 41.1, 33.7, 30.5, 30.5, 27.7, 27.7, 26.5, 26.4. FTIR (thin film, cm⁻¹) 1775 (m), 1656 (m), 1424 (m), 1191 (s), 1137 (s), 720 (w). HRMS (ESI, m/z) calculated for C₃₅H₄₃ClN₃O₈ [M⁺H]⁺: 668.2733; found: 668.2720.

### Rhodol Tert-Butyl Ester (S5)

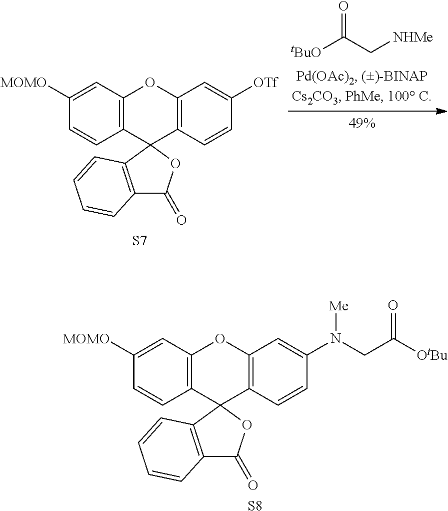

A 10 mL pressure vessel was sequentially charged with freshly free-based sarcosine tert-butyl ester (295.8 mg, 2.04 mmol, 1.50 equiv), rhodol triflate S7 (690.0 mg, 1.36 mmol, 1 equiv), cesium carbonate (663.7 mg, 2.04 mmol, 1.50 equiv), (±)-2,2′-bis(diphenylphosphino)-1,1′-binaphthyl (BINAP, 254.1 mg, 408 μmol, 0.30 equiv), and palladium(II) acetate (61.1 mg, 272 μmol, 0.20 equiv). Toluene (6.90 mL) was added to the reaction mixture and sparged with nitrogen. After 5 min, the vessel was quickly sealed and the reaction was stirred at room temperature. After 30 min, the reaction was heated to 100° C. After 4 h, the reaction was cooled to room temperature and purified directly by flash column chromatography on silica gel (eluent: 25% ethyl acetate in hexanes) to afford rhodol tert-butyl ester S8 (337 mg, 670 μmol, 49%) as a white foam.

¹H NMR (500 MHz, CDCl₃) δ 8.00 (d, J=7.6 Hz, 1H), 7.64 (t, J=7.5 Hz, 1H), 7.59 (t, J=7.4 Hz, 1H), 7.16 (d, J=7.6 Hz, 1H), 6.94 (s, 1H), 6.67 (s, 2H), 6.60 (d, J=8.8 Hz, 1H), 6.47 (d, J=2.7 Hz, 1H), 6.34 (dd, J=8.9, 2.7 Hz, 1H), 5.18 (s, 2H), 3.95 (s, 2H), 3.47 (s, 3H), 3.07 (s, 3H), 1.43 (s, 9H). ¹³C NMR (126 MHz, CDCl₃) δ 169.8, 169.7, 158.8, 153.3, 152.8, 152.7, 151.0, 134.9, 129.6, 129.2, 129.0, 127.2, 125.0, 124.2, 112.9, 112.7, 108.8, 107.2, 103.7, 98.9, 94.4, 84.0, 82.1, 56.3, 55.1, 39.8, 28.2. FTIR (thin film, cm⁻¹) 2975 (br, m), 2929 (br, m), 1762 (s), 1614 (s), 1520 (w), 1503 (w), 1426 (m), 1250 (m), 1154 (s), 1107 (s), 1004 (m). HRMS (ESI, m/z) calculated for C₂₄H₂₀NO₆ [M⁺H]⁺: 504.2017; found: 504.2007. TLC (30% ethyl acetate in hexanes), Rf: 0.31 (UV).

### HaloTag Rhodol (9)

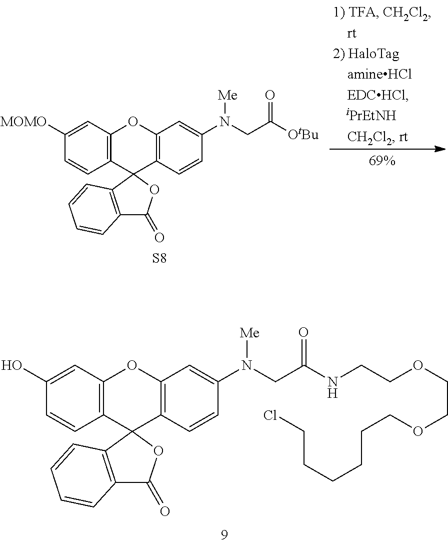

Trifluoroacetic acid (2 mL) was added in one portion to a solution of tert-butyl ester S8 (43.2 mg, 85.8 μmol, 1 equiv) in dichloromethane (2 mL) at room temperature. After 1 h, the reaction mixture was concentrated under reduced pressure to afford a red oil. HaloTag amine hydrochloride (67.0 mg, 257 μmol, 3.00 equiv), N-(3-dimethylaminopropyl)-N′-ethylcarbodiimide hydrochloride (EDC·HCl, 164.5 mg, 858 μmol, 10.0 equiv), and dichloromethane (5 mL) were sequentially added to the oil. N,N-diisopropylethylamine (299 μL, 1.72 mmol, 20.0 equiv) was then added to the solution at room temperature. After 2 h, the reaction was concentrated to dryness under reduced pressure and purified by automated flash column chromatography on a C₁₈ column (Biotage, KP-C18-HS, 30 g, 50 mL/min; time (min), % MeCN in H₂O+0.1% TFA: 0, 0; 100, 13.5). The column fractions containing HaloTag rhodol 9 were concentrated under reduced pressure then purified by preparative HPLC on a C₁₈ column (20 mL/min, time (min), % MeCN in H₂O+0.1% TFA: 0, 0; 60, 100) to afford pure HaloTag rhodol 9 (36.3 mg, 59.6 μmol, 69%) as a red oil.

¹H NMR (500 MHz, CD₃OD) δ 8.34 (d, J=7.4 Hz, 1H), 7.86 (dt, J=7.4, 1.5 Hz, 1H), 7.82 (dt, J=7.6, 1.4 Hz, 1H), 7.41 (d, J=7.4 Hz, 1H), 7.23 (dd, J=12.4, 9.8 Hz, 2H), 7.12 (dd, J=11.5, 2.0 Hz, 2H), 7.04 (d, J=2.5 Hz, 1H), 6.98 (dd, J=8.8, 2.4 Hz, 1H), 4.42 (s, 2H), 3.64-3.50 (m, 8H), 3.48-3.39 (m, 4H), 3.36 (s, 3H), 3.35 (s, 1H), 1.80-1.66 (m, 2H), 1.61-1.50 (m, 2H), 1.49-1.39 (m, 2H), 1.39-1.30 (m, 2H). ¹³C NMR (126 MHz, CD₃OD) δ 169.5, 168.9, 168.1, 160.2, 159.6, 158.4, 136.6, 134.2, 132.7, 132.6, 132.0, 131.8, 131.8, 130.8, 118.5, 117.2, 116.5, 116.3, 103.3, 98.3, 72.2, 71.2, 71.2, 70.3, 56.7, 49.8, 45.7, 41.3, 40.5, 33.7, 30.5, 27.7, 26.4. FTIR (thin film, cm⁻¹) 2939 (br, m), 2866 (br, m), 1592 (s), 1498 (m), 1406 (m), 1323 (w), 1278 (w), 1186 (s), 1131 (s). HRMS (ESI, m/z) calculated for C₃₃H₃₈ClN₂O₇ [M⁺H]⁺: 609.2362; found: 609.2352.

### HaloTag Rhodol N-Oxide (8)

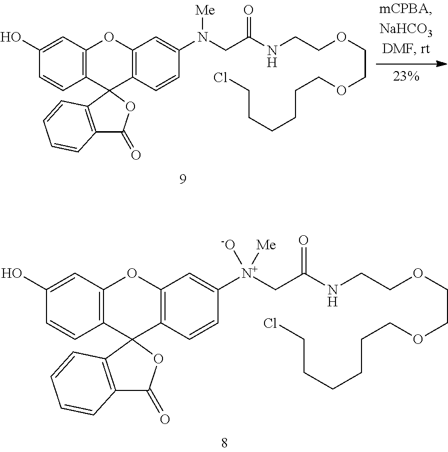

Sodium bicarbonate (57.9 mg, 690 μmol, 10.0 equiv) and m-chloroperbenzoic acid (39.5 mg, 345 μmol, 5.0 equiv) were added sequentially to a solution of HaloTag rhodol 9 (42.0 mg, 69.0 μmol, 1 equiv) in N,N-dimethylformamide (1 mL) at room temperature. After 30 m, the solution was cooled to −78° C., and N,N-diisopropylethylamine (120.1 μL, 690 μmol, 10.0 equiv) was added. The reaction mixture was then diluted with ice-water (10 mL), and immediately allowed to warm to room temperature over 30 min. The solution was purified by automated flash column chromatography on a C₁₈ column (Biotage SNAP KP-C18-HS, 30 g, 50 mL/min, time (min), % MeCN in H₂O: 0, 0; 4.5, 0; 18, 100). Column fractions containing HaloTag rhodol N-oxides 8 were collected and concentrated under reduced pressure. This mixture was purified by preparative HPLC on a C₁₈ column (20 mL/min, time (min), % MeCN in H₂O+0.1% TFA: 0, 0; 60, 100) to afford an inseparable diastereomeric mixture of HaloTag rhodol N-oxides 8 (9.8 mg, 15.7 μmol, 23%) as a light pink film.

¹H NMR (500 MHz, CD₃OD) δ 8.12-8.01 (m, 2H), 7.83-7.69 (m, 2H), 7.69-7.56 (m, 1H), 7.28-7.17 (m, 1H), 7.03-6.92 (m, 1H), 6.78-6.70 (m, 1H), 6.69-6.62 (m, 1H), 6.62-6.54 (m, 1H), 4.53 (dd, J=14.4, 2.9 Hz, 1H), 4.42 (d, J=14.5 Hz, 1H), 3.72-3.62 (m, 3H), 3.58-3.45 (m, 6H), 3.45-3.37 (m, 4H), 3.21-3.11 (m, 2H), 1.78-1.68 (m, 2H), 1.58-1.48 (m, 2H), 1.48-1.38 (m, 2H), 1.38-1.30 (m, 2H). ¹³C NMR (126 MHz, CD₃OD) δ 170.9, 165.7, 161.6, 154.9, 154.2, 153.5, 152.8, 152.7, 136.8, 131.5, 131.5, 130.5, 130.4, 130.2, 127.7, 126.1, 125.2, 122.0, 121.9, 117.3, 117.1, 114.4, 111.8, 111.5, 110.6, 103.6, 73.5, 72.2, 72.2, 71.4, 71.3, 71.2, 71.1, 70.4, 70.4, 63.3, 63.2, 45.7, 39.9, 33.7, 33.7, 30.5, 27.7, 26.5. FTIR (thin film, cm⁻¹) 2934 (br, s), 2863 (br, s), 1766 (s), 1674 (m), 1609 (m), 1425 (m), 1284 (w), 1107 (s), 762 (w). HRMS (ESI, m/z) calculated for C₃₃H₃₈ClN₂O₈ [M⁺H]⁺: 625.2311; found: 625.2300.

### 4,4,4′,5,5′,5′-Hexamethyl-4,5-diphenyl-2,2′-bi-1,3,2-dioxaborolane (S1)

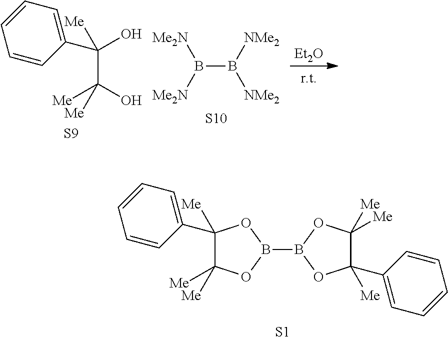

Tetrakis(dimethylamino)diboron (S10, 29.7 μL, 139 μmol, 0.500 equiv) was added to pinacol derivative (S9, 50.0 mg, 277 μmol, 1 equiv) in diethyl ether (1 mL) at room temperature. After 2 h 40 min, the reaction was concentrated to dryness by passing a stream of nitrogen gas over the solution. The residue was dissolved in minimal DMF and purified by preparative HPLC on a C₁₈ column (20 mL/min, time (min), % MeCN in H₂O+0.1% TFA: 0, 0; 5, 0; 35, 100). Only the pure fractions were collected and concentrated on a rotary evaporator to yield the major diastereomer of diboron S1 (9.4 mg, 24.9 μmol, 18%) as a white solid. The pure fractions of the minor diastereomer of diboron S1 (5.4 mg, 14.3 μmol, 10%) was also isolated as a white solid. The major diastereomer was fully characterized and used in further kinetics studies.

¹H NMR (500 MHz, CDCl₃) δ 7.51-7.39 (m, 2H), 7.39-7.31 (m, 2H), 7.31-7.26 (m, 1H), 1.63 (s, 3H), 1.57 (s, 3H), 0.84 (s, 3H). ¹³C NMR (126 MHz, CDCl₃) δ 143.1, 128.2, 127.2, 125.0, 87.8 (d, J=5.2 Hz), 84.9 (d, J=5.6 Hz), 28.5, 27.0, 24.5. ¹¹B NMR (160 MHz, CDCl₃) δ 31.1. FTIR (thin film, cm⁻¹) 2978 (w), 2926 (w), 1456 (w), 1372 (w), 1283 (m), 1264 (m), 1169 (m), 1139 (m), 701 (m). HRMS (ESI, m/z) calculated for C₂₂H₂₈B₂NaO₄ [M+Na]⁺: 401.2066; found: 401.2062.

### Example 3: Kinetics of Diboron Hydrolysis

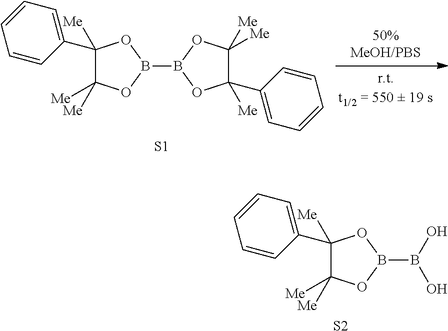

A solution of diboron S1 (17.5 mM in MeOH, 50.0 μL, 875 nmol) was added to MeOH (450 μL). A solution of BnPh₃PCl (100 mM in PBS, 1.00 μL, 100 nmol) was then added. PBS (500 μL) was then introduced, and the resulting solution was quickly filtered through a 0.45 μm PTFE syringe filter. The solution (75 μL) was immediately analyzed by HPLC on a C₁₈ analytical column (4.6×50 mm, 0.7 mL/min, time (min), % MeCN in H₂O+0.1% TFA: 0, 0; 2.5, 54; 3.5, 80; 6, 100; 6.01, 50; 7.2, 50). The solution was sampled every 8 min 45 s (525 s). Peak integrals for diboron S1 were normalized against that of the BnPh₃PCl internal standard.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for chemoselective modification of a target molecule comprising an amine N-oxide in a biological sample, the method comprising:
selectively reacting the amine N-oxide group of the target molecule with a boron agent, wherein the reacting reduces the amine N-oxide to an amine to produce a modified target molecule.

2. The method of claim 1, wherein the modified target molecule is an activated target molecule.

3. The method of claim 1, wherein the modified target molecule is cleaved to produce a first target fragment and a second target fragment.

4. The method of any one of claims 1-3, wherein the target molecule comprises a profluorophore and the reacting activates the profluorophore to produce a fluorescent target molecule.

5. The method of any one of claims 1-3, wherein the target molecule comprises a prodrug and the reacting activates the prodrug to produce a drug.

6. The method of any one of claims 1 and 3, wherein the reacting modifies an amine N-oxide-linker of the target molecule to produce a cleavable amine-linker.

7. The method of claim 6, wherein the target molecule comprises a biomolecule covalently linked via the amine N-oxide-linker to a chemical entity and the method further comprises cleaving the cleavable amine-linker to release the chemical entity.

8. The method of claim 7, wherein the chemical entity is a drug or a detectable label.

9. The method of any one of claims 6-8, wherein the amine N-oxide-linker comprises a self immolative linker group.

10. The method of any one of claims 6-9, wherein the amine N-oxide-linker comprises a group selected from the group consisting of para-amino-benzyloxycarbonyl (PABC), meta-amino-benzyloxycarbonyl (MABC), para-amino-benzyloxy (PABO), meta-amino-benzyloxy (MABO) and para-aminobenzyl.

11. The method of any one of claims 1-10, wherein the target molecule comprises a biomolecule.

12. The method of claim 11, wherein the biomolecule is a protein.

13. The method of claim 12, wherein the biomolecule is an antibody.

14. The method of any one of claims 1-13, wherein the boron agent has the formula 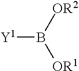
wherein:
Y¹ is an aryl, a substituted aryl, a heteroaryl, a substituted heteroaryl, an alkyl, a substituted alkyl or —B(OR⁶)(OR⁷);
R¹, R², R⁶ and R⁷ are each independently H, an alkyl, a substituted alkyl, an aryl, a substituted aryl, a heteroaryl or a substituted heteroaryl, and
R¹ and R² or R⁶ and R⁷ may be optionally cyclically linked.

15. The method of claim 14, wherein Y¹ is —B(OR⁶)(OR⁷).

16. The method of any one of claims 1-14, wherein the boron agent is a diboron agent.

17. The method of any one of claims 15-16, wherein the boron agent is bis(pinacolato)diboron ((Bpin)₂) or bis(catecholato)diboron).

18. The method of any one of claims 1-17, wherein the biological sample comprises a cell, a cell lysate, a tissue, or a fluid.

19. The method of any one of claims 1-18, wherein the biological sample is in vivo.

20. A composition, comprising:
a target molecule comprising an amine N-oxide; and
a boron agent;
contained in a biological sample.

21. A conjugate, comprising a first target molecule and a second target molecule, covalently linked via an amine N-oxide-linker.

22. The conjugate of claim 21, wherein the first target molecule is a biomolecule.

23. The conjugate of claim 22, wherein the second target molecule is a biomolecule.

24. The conjugate of claim 22, wherein the second target molecule is a chemical entity.

25. The conjugate of claim 24, wherein the chemical entity is a drug or a detectable label.

26. The conjugate of claim 25, wherein the biomolecule is an antibody or an antibody fragment and the chemical entity is a chemotherapeutic drug.

27. The conjugate of any one of claims 21-26, wherein the amine N-oxide-linker comprises a self immolative linker.

28. The conjugate of any one of claims 21-27, wherein the amine N-oxide-linker comprises a group selected from the group consisting of para-amino-benzyloxycarbonyl (PABC), meta-amino-benzyloxycarbonyl (MABC), para-amino-benzyloxy (PABO), meta-amino-benzyloxy (MABO) and para-aminobenzyl.
